(19) United States
(12) Patent Application Publication
(10) Pub. No.: US 2026/0258381 A1
(43) Pub. Date: Sep. 3, 2026

(54) LIPASE WITH IMPROVED STEREOSELECTIVITY AND LIPASE-BASED CHIRAL RESOLUTION METHODS

(71) Applicant: Bayer Aktiengesellschaft, Leverkusen (DE)

(72) Inventors: Elia Calderini, Köln (DE); Alba Hernandez Martin, Düsseldorf (DE); Oliver Kensch, Pulheim (DE); Florian Richter, Köln (DE); Christian Pitzler, Köln (DE); Wahed Ahmed Moradi, Monheim (DE); Andreas Rembiak, Langerwehe (DE); Mark James Ford, Oberreifenber (DE); Anton Lishchynskyi, Solingen (DE); Markus Spelberg, Düsseldorf (DE); Nadine Zumbrägel, Erftstadt (DE); Fabian Basso, Köln (DE); Florian Erver, Wiesbaden (DE)

(21) Appl. No.: 19/164,670

(22) PCT Filed: Mar. 7, 2024
(86) PCT No.: PCT/EP2024/055979

§ 371 Date: Sep. 12, 2025

(30) Foreign Application Priority Data
Mar. 14, 2023 (EP) 23161854.7

(51) Int. Cl. C12N 9/20; C12N 15/52; C12P 7/42; C12P 41/00

(52) U.S. Cl. CPC C12N 9/20; C12N 15/52; C12P 7/42; C12P 41/005; C12Y 301/01003

(57) ABSTRACT

The present invention relates to proteins having improved lipase activity, nucleic acid molecules encoding respective proteins having improved lipase activity and methods for chiral resolution of secondary alcohols.

### TECHNICAL FIELD

The present invention relates to proteins with improved lipase activity, nucleic acid molecules encoding said proteins as well as enzymatic methods for the chiral resolution of secondary alcohols.

### BACKGROUND

Enantiomerically enriched or pure alcohols are important compounds for the production of agrochemical or pharmaceutical compounds. Thus, the absolute configuration of the stereocenters of chiral alcohols is crucial for the synthesis of the corresponding active agents. In the production of a desired target molecule, generation of correct chirality is often a challenge.

Various methods for preparing enantiomerically enriched alcohols are known. On the one hand, chiral transition metal catalysts can be used, which, however, are characterized by high costs (G. R. Cook, Transition Metal-Mediated Kinetic Resolution, Current Organic Chemistry, 2000, 4, 869-885; Hirama et al. J. Org. Chem. 1988, 53, 708). Besides metal catalysis, an organocatalytic acyl transfer kinetic resolution is described for bulky ester substrates (Deng et al. J. Org. Chem. 2015, 80, 6, 3159). Also known is the use of enzymes, for example lipases, as biocatalysts for the production of chiral compounds. Kirchner et al. (J. Am. Chem. Soc. (1985), 107, 7072-7076) report two lipases that act as highly stereoselective, practical catalysts in nearly anhydrous organic solvents. Under such “unnatural” conditions the enzymes can asymmetrically catalyze reactions of esterification and transesterification which are not feasible in aqueous solutions because of the domination of hydrolysis. As a result, a number of optically active alcohols, carboxylic acids, and esters have been prepared on a gram scale. Faber and Riva (1992; Synthesis 1992 (10), 895-910), and Nascimento et al. (2003, Tetrahedron Asymmetry 14, 311-311) describe enzymatic resolution reactions of secondary alcohols by transesterification. In addition, Patil et al. (J. Org. Chem. 2008, 73, 4476-4483) and Reddy et. al (Synth. Communications, 2003, 33, 3717-3726) showed the use of esterases for the enantioselective ester hydrolysis in buffered aqueous systems, however in the presence of DMSO and only moderate yields.

U.S. Pat. No. 4,732,853 A describes a method of making chiral epoxy alcohols by means of enantioselective hydrolysis with lipases.

EP 0716712 B1 describes the lipase-catalyzed acylation of alcohols with diketenes, especially for the production of enantioselective acylated alcohols from racemic alcohols.

WO2012146935A1 discloses modified lipase variants, as well as polynucleotides and recombinant expression vectors encoding the lipase variant polypeptides, as well as methods for producing such lipase variants in selected bacterial and fungal host cells. The specified lipase variants have increased enzyme specificity or enhanced trans-selectivity. Further described are methods of their use for reducing or eliminating trans-fatty acids from substrates.

Although several improvements of lipases have been achieved so far, several limitations arising during the asymmetric synthesis of secondary alcohols or resolution of racemic alcohols still have to be overcome, such as unfavorable equilibrium, substrate and product inhibition, poor thermostability, insufficient substrate specificity and in particular low enantioselectivity of the lipase. In addition, downstream processing and isolation of the target chiral alcohols are challenging within large scale applications while importance of atom economy is largely increased on industrial scale.

Thus, there is a need for further improvement of lipases, in particular with respect to the production of enantiomerically enriched or pure products with increased atom economy, as well as for further process improvements.

### SUMMARY

The present invention solves the above-described problems at least partially by providing novel lipases with superior enantiomer selectivity as well as providing novel methods for the chiral resolution of industrially relevant building blocks.

The lipases described herein have certain advantages over known wild-type and other already known lipases. In particular, the modified or variant lipases described herein have the advantage that they can produce enantiomerically enriched or enantiomerically nearly pure or pure compounds better than respective wild-type lipases.

A first aspect of the present invention relates to a protein having the activity of a lipase wherein the protein is encoded by an amino acid sequence having at least 80%, preferably 85%, more preferably 90%, furthermore preferably 92%, even more preferably 95% with the amino acid sequence shown under SEQ ID No. 1,
characterized in that the amino acid sequence of the variant differs from the amino acid sequence of SEQ ID NO. 1 in at least one of the following positions:
i. the amino acid at position 44 is different from L, preferably the amino acid at position 44 is M, W or Y;
ii. the amino acid at position 51 is different from F, preferably the amino acid at position 51 is N, or M;
iii. the amino acid at position 52 is different from V, preferably the amino acid at position 52 is L;
iv. the amino acid at position 53 is different from T, preferably the amino acid at position 53 is S, P, I, E, or A;
v. the amino acid at position 54 is different from D, preferably the amino acid at position 54 is Q, M, F, G, E, L, T or P;
vi. the amino acid at position 55 is different from A, preferably the amino acid at position 55 is R, M, D, Y, S, or I;
vii. the amino acid at position 109 is different from G, preferably the amino acid at position 109 is H or F;
viii. the amino acid at position 110 is different from M, preferably the amino acid at position 110 is T or V;
ix. the amino acid at position 111 is different from A, preferably the amino acid at position 111 is T or S;
x. the amino acid at position 117 is different from Y, preferably the amino acid at position 117 is F or S;
xi. the amino acid at position 121 is different from Y, preferably the amino acid at position 121 is V;
xii. the amino acid at position 122 is different from K, preferably the amino acid at position 122 is Q, A, Y, R, or V;
xiii. the amino acid at position 153 is different from H, preferably the amino acid at position 153 is N, Y, D, E, or C;
xiv. the amino acid at position 160 is different from T, preferably the amino acid at position 160 is E, C, D, P, I, Q, K, M, S, F, A, or N;
xv. the amino acid at position 179 is different from D, preferably the amino acid at position 179 is C;
xvi. the amino acid at position 181 is different from A, preferably the amino acid at position 181 is Q;
xvii. the amino acid at position 184 is different from A, preferably the amino acid at position 184 is G or T;
xviii. the amino acid at position 211 is different from Y, preferably the amino acid at position 211 is E;
xix. the amino acid at position 212 is different from A, preferably the amino acid at position 212 is S or P;
xx. the amino acid at position 216 is different from Y, preferably the amino acid at position 216 is K or A;
xxi. the amino acid at position 234 is different from S, preferably the amino acid at position 234 is K, T or G;
xxii. the amino acid at position 235 is different from S, preferably the amino acid at position 235 is V or M;
xxiii. the amino acid at position 236 is different from K, preferably the amino acid at position 236 is T;
xxiv. the amino acid at position 238 is different from R, preferably the amino acid at position 238 is A, K, D, E, or Q;
xxv. the amino acid at position 240 is different from Y, preferably the amino acid at position 240 is F;
xxvi. the amino acid at position 289 is different from D, preferably the amino acid at position 289 is S or G;
xxvii. the amino acid at position 291 is different from G, preferably the amino acid at position 291 is E or W;
xxviii. the amino acid at position 317 is different from N, preferably the amino acid at position 317 is T;
xxix. the amino acid at position 320 is different from N, preferably the amino acid at position 320 is E or G;
xxx. the amino acid at position 321 is different from L, preferably the amino acid at position 321 is F.

SEQ ID NO.1 refers to a reference protein sequence with lipase activity.

The meaning of amino acid abbreviations A, C, D, E, F, G, H, I, K, L, M, N, P, Q, R, S, T, V, W, Y is derivable herein below in Table 2 under the paragraph sub-titled “Description of the Sequences”.

An “amino acid corresponding to position x” in a first amino acid sequence (e.g. position 44 in SEQ ID No. 1) means herein that an amino acid of a second amino acid sequence, when compared with the first amino acid sequence, appears at position x of the first amino acid sequence in a pairwise sequence alignment of the first amino acid sequence with the second amino acid sequence in case the numbering of the amino acids of the second amino acid sequence differs from the amino acid numbering of the first amino acid sequence.

In the context of the present invention, the term “identity” in respect to sequence identity or sequences being identical to is to be understood as meaning the number of identical amino acids or nucleotides shared over the entire sequence length by a first nucleic or amino acid sequence with another (second) nucleic or amino acid sequence, respectively, expressed in percent.

“Sequence identity” can be determined by alignment of two amino acid or two nucleotide sequences using global or local alignment algorithms comprised for example in known software like GAP or BESTFIT or the Emboss program “Needle”. This software uses the Needleman and Wunsch global alignment algorithm for aligning two sequences, over their entire length, maximizing the number of matches and minimizing the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using software, such as EMBOSS, accessible at world wide web site of the EBI (ebi.ac.uk/Tools/emboss/). Alternatively, sequence similarity or identity may be determined by searching against databases (e.g. EMBL, GenBank) by using commonly known algorithms and output formats such as FASTA, BLAST, etc., but preferably hits should be retrieved and aligned pairwise to finally determine sequence identity.

If sequences to be compared with one another are of different length, the identity is to be determined by determining the identity in percent of the number of amino acids or nucleotides, respectively, which the shorter sequence shares with the longer sequence. Preferably, the identity is determined using the known and publicly available computer program ClustalW (Thompson et al., Nucleic Acids Research 22 (1994), 4673-4680). ClustalW is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany. ClustalW can also be downloaded from various Internet pages, inter alia from IGBMC (Institut de Génétique et de Biologie Moléculaire et Cellulaire, B.P.163, 67404 Illkirch Cedex, France; ftp://ftp-igbmc.u-strasbg.fr/pub/) and from EBI (ftp://ftp.ebi.ac.uk/pub/software/) and all mirrored Internet pages of the EBI (European Bioinformatics Institute, Wellcome Trust Genome Campus, Hinxton, Cambridge CB10 1SD, UK).

Preferably, use is made of the ClustalW computer program of version 1.8 or Clustal 2 to determine the identity between proteins described in the context of the present invention and other proteins. Here, the parameters have to be set as follows: KTUPLE=1, TOPDIAG=5, WINDOW-5, PAIRGAP=3, GAPOPEN=10, GAPEXTEND=0.05, GAPDIST=8, MAXDIV=40, MATRIX=GONNET, ENDGAPS(OFF), NOPGAP, NOHGAP.

Preferably, use is made of the ClustalW computer program of version 1.8 or Clustal 2 to determine the identity for example between the nucleotide sequence of the nucleic acid molecules described in the context of the present invention and the nucleotide sequence of other nucleic acid molecules. Here, the parameters have to be set as follows:
KTUPLE=2, TOPDIAGS=4, PAIRGAP=5, DNAMATRIX: IUB, GAPOPEN=10, GAPEXT=5, MAXDIV=40, TRANSITIONS: unweighted.

“Identity” furthermore means that there is a functional and/or structural equivalence between the nucleic acid molecules in question or the proteins encoded by them. Functional equivalence means that the nucleic acid molecule sequences or the amino acid sequences encode a protein having the activity of a lipase. The nucleic acid molecules which are homologous to the molecules described above and represent derivatives of these molecules are generally variants of these molecules which represent modifications having the same biological function or catalyzing the same reaction, i.e. coding for a protein having the activity of a lipase. They may be either naturally occurring variants, for example sequences from other species, or mutations, where these mutations may have occurred in a natural manner or were introduced by targeted mutagenesis. Furthermore, the variants may be synthetically produced sequences. The allelic variants may be either naturally occurring variants or synthetically produced variants or variants generated by recombinant DNA techniques. However, concerning the present invention it is decisive that those variants encode proteins having lipase-activity and comprise the amino acid substitutions (replacements), deletions or insertions described herein concerning the proteins according to the invention.

A special type of derivatives are, for example, nucleic acid molecules which differ from the nucleic acid molecules described in the context of the present invention as a result of the degeneracy of the genetic code.

According to the NC-IUBMB (Nomenclature Committee of the International Union of Biochemistry and Molecular Biology) lipases belong to the class of hydrolases (EC 3). Hydrolase is a class of enzymes that commonly perform as biochemical catalysts that use water to break a chemical bond, which typically results in dividing a larger molecule into smaller molecules. Under unnatural, anhydrous, conditions these enzymes can also catalyze reactions of esterification, e.g. acetylation, and transesterification. The group of hydrolases comprises enzymes acting on ester bonds (EC 3.1) encompassing carboxylic ester hydrolases (EC 3.1.1) and as a subgroup lipases (EC 3.1.1.3). Lipases have been identified from plants, mammals and microorganisms including e.g. Pseudomonas, Vibrio, Acinetobacter, Burkholderia, Chromobacterium, cutinase from Fusarium solani (FSC), Candida antarctica A (CalA), Rhizopus oryzae (ROL), Thermomyces lanuginosus (TLL), Rhizomucor miehei (RML), Aspergillus Niger, Fusarium heterosporum, Fusarium oxysporum or Fusarium culmorum.

If a protein has the activity of a lipase, this can be detected with methods known and described in the art.

It is not decisive which method is used for detecting if a protein according to the invention has the activity of a lipase. Preferably, in connection with the present invention, the method is described in the “Example”-section.

In a further embodiment of the invention, the amino acid sequence of the variant differs from the amino acid sequence of SEQ ID NO. 1 by at least one of the following modifications:
i. the amino acid at position 44 is W or Y;
ii. the amino acid at position 54 is F;
iii. the amino acid at position 55 is R;
iv. the amino acid at position 109 is H;
V. the amino acid at position 110 is T or V;
vi. the amino acid at position 117 is F;
vii. the amino acid at position 122 is Q or R;
viii. the amino acid at position 160 is E;
ix. the amino acid at position 216 is K;
x. the amino acid at position 236 is T;
xi. the amino acid at position 238 is K or E;
xii. the amino acid at position 240 is F.

Preferably, the amino acid sequence of the variant differs from the amino acid sequence of SEQ ID NO. 1 by at least the mutation G109H, i.e. the amino acid at position 109 is H.

Lipase variant proteins according to the invention may exhibit further amino acid modifications (amino acid substitutions, deletions or insertions) compared to the amino acid sequences described herein above in respect to the amino acid sequence shown under SEQ ID No. 1.

In a further embodiment of the invention, the amino acid sequence of the variant differs from the amino acid sequence of SEQ ID NO. 1 by at least two, further preferably at least three, even further preferably at least four, in particular preferably at least five of the following modifications selected from:
i. the amino acid at position 57 is different from N, preferably the amino acid is P;
ii. the amino acid at position 109 is different from G, preferably the amino acid is H;
iii. the amino acid at position 122 is different from K, preferably the amino acid is R;
iv. the amino acid at position 212 is different from A, preferably the amino acid is P;
V. the amino acid at position 234 is different from S, preferably the amino acid is K;
vi. the amino acid at position 289 is different from D, preferably the amino acid is G.

Preferably, the amino acid sequence of the variant differs from the amino acid sequence of SEQ ID NO. 1 by the following modifications:
i. the amino acid at position 57 is P;
ii. the amino acid at position 109 is H;
iii. the amino acid at position 212 is P;
iv. the amino acid at position 234 is K; and
v. the amino acid at position 289 is G.

Further preferably, the amino acid sequence of the variant differs from the amino acid sequence of SEQ ID NO. 1 by the following modifications:
i. the amino acid at position 57 is P;
ii. the amino acid at position 109 is H;
iii. the amino acid at position 122 is R;
iv. the amino acid at position 212 is P;
V. the amino acid at position 234 is K; and
vi. the amino acid at position 289 is G.

In a further embodiment of the invention, the amino acid sequence of the variant differs from the amino acid sequence of SEQ ID NO. 1 by the following modifications:
the amino acid at position 57 is P; the amino acid at position 109 is H; the amino acid at position 122 is R or K or Q, preferably R; the amino acid at position 212 is P; the amino acid at position 234 is K; the amino acid at position 289 is G;
and additionally at least one of the following modifications: the amino acid at position 44 is W or Y; the amino acid at position 54 is F, the amino acid at position 55 is R, the amino acid at position 110 is V or T; the amino acid at position 111 is T, the amino acid at position 117 is F, the amino acid at position 160 is E, the amino acid at position 216 is K, the amino acid at position 236 is T, the amino acid at position 238 is K or E, the amino acid at position 240 is F.

Preferred embodiments of the invention are proteins according to the invention encoding lipases having the amino acid sequences shown under SEQ ID Nos. 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239.

One further embodiment of the invention concerns nucleic acid molecules encoding a protein according to the invention.

Nucleic acid molecules according to the invention can be any kind of nucleic acid, as long as the nucleic acid encodes a protein according to the invention. The nucleic acids can be ribonucleic nucleic acid molecules (e.g. RNA, mRNA) or deoxyribonucleic nucleic acid molecules (DNA, including genomic DNA which may or may not comprise introns and coding DNA).

Of particular interest for the invention are nucleic acid molecules encoding proteins having the activity of a lipase comprising the amino acid sequences shown under SEQ ID Nos. 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240.

The invention therefore also concerns nucleic acid molecules encoding a protein having the activity of a lipase selected from the group consisting of
a) nucleic acid molecules comprising the nucleic acid sequences shown under SEQ ID Nos. 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240;

b) nucleic acid molecules having at least 60%, preferably 70%, more preferably 80%, furthermore preferably 90%, even more preferably 95%, even furthermore preferably 96%, particular preferably 97%, most preferably 98% or especially preferably 99% identity with the nucleic acid sequences shown under a).

In the context of the present invention, the term “hybridizing with” means hybridization under conventional hybridization conditions, preferably under stringent conditions, as described, for example, in Sambrook et al. (Molecular Cloning, A Laboratory Manual, 3rd edition (2001) Cold Spring Harbor Laboratory Press, Cold Spring Harbor, NY. ISBN: 0879695773) or Ausubel et al. (Short Protocols in Molecular Biology, John Wiley & Sons; 5th edition (2002), ISBN: 0471250929). With particular preference, “hybridization” means a hybridization under the following conditions:
hybridization buffer:
2×SSC; 10×Denhardt solution (Fikoll 400+PEG+BSA; ratio 1:1:1); 0.1% SDS; 5 mM EDTA; 50 mM Na2HPO4; 250 μg/ml of herring sperm DNA; 50 μg/ml of tRNA;
or
25 M sodium phosphate buffer pH 7.2; 1 mM EDTA; 7% SDS
hybridization temperature: T=65 to 68° C.
wash buffer: 0.1×SSC; 0.1% SDS
wash temperature: T=65 to 68° C.

Nucleic acid molecules which hybridize with nucleic acid molecules coding for a protein having the activity of a lipase may originate from any organism; accordingly, they may originate from bacteria, fungi, animals, humans, plants or viruses.

Nucleic acid molecules which hybridize with nucleic acid molecules coding for a protein having the activity of a lipase preferably originate from microorganisms, more preferably from fungi or bacteria, most preferably from bacteria.

Nucleic acid molecules which hybridize with the molecules mentioned may be isolated, for example, from genomic or from cDNA libraries. Such nucleic acid molecules can be identified and isolated using the nucleic acid molecules described herein or they can be identified and isolated using parts of these molecules or the reverse complements of these molecules, for example by hybridization according to standard methods (see, for example, Sambrook et al., Molecular Cloning, A Laboratory Manual, 3rd edition (2001) Cold Spring Harbor Laboratory Press, Cold Spring Harbor, NY. ISBN: 0879695773; Ausubel et al., Short Protocols in Molecular Biology, John Wiley & Sons; 5th edition (2002), ISBN: 0471250929) or by amplification using PCR.

The fragments used as hybridization samples may also be synthetic fragments or oligonucleotides prepared using the customary synthesis techniques, whose sequence is essentially identical to the nucleic acid molecule described in the context of the present invention. Once genes which hybridize with the nucleic acid sequences described in the context of the present invention are identified and isolated, the sequence should be determined and the properties of the proteins coded for by this sequence should be analyzed to determine whether they are proteins having the activity of a lipase. Methods of how to determine whether a protein has the activity of a protein having the activity of a lipase are known to the person skilled in the art.

The molecules hybridizing with the nucleic acid molecules described in the context of the present invention comprise in particular fragments, derivatives and allelic variants of the nucleic acid molecules mentioned. In the context of the present invention, the term “derivative” means that the sequences of these molecules differ in one or more positions from the sequences of the nucleic acid molecules described above and are highly identical to these sequences. The differences to the nucleic acid molecules described above may, for example, be due to deletion, addition, substitution, insertion or recombination.

The meaning of nucleotide abbreviations a, c, g, t, and those of abbreviations for degenerate nucleotides r, y, s, w, k, m, b, d, h, v, n is derivable herein below from Table 1 under the paragraph sub-titled “Description of the Sequences”. Which amino acids are encoded by codons comprising degenerate nucleotides is derivable herein below from Table 3 under the paragraph sub-titled “Description of the Sequences”.

Furthermore, the invention relates to recombinant nucleic acid molecules comprising a nucleic acid molecule according to the invention.

In conjunction with the present invention, the term “recombinant nucleic acid molecule” is to be understood to mean a nucleic acid molecule, which contains additional sequences in addition to nucleic acid molecules according to the invention, which do not naturally occur in the combination in which they occur in recombinant nucleic acids according to the invention. Here, the abovementioned additional sequences can be any sequences, preferably they are functional or regulatory sequences (promoters, termination signals, enhancers, ribosome binding sites (rbs), leader sequences enhancing transcription, translation or RNA stability, subcellular targeting sequences etc.), particularly preferably they are functional or regulatory sequences that are active in microorganisms, and especially particularly preferably they are regulatory sequences that are active in fungi, in particular yeasts or in bacteria. Methods for the creation of recombinant nucleic acid molecules according to the invention are known to the person skilled in the art and include genetic methods such as bonding nucleic acid molecules by way of ligation, genetic recombination, or new synthesis of nucleic acid molecules. Those methods are described e.g. in Sambrok et al. (Molecular Cloning, A Laboratory Manual, 3rd edition (2001) Cold Spring Harbour Laboratory Press, Cold Spring Harbour, NY. ISBN: 0879695773) or Ausubel et al. (Short Protocols in Molecular Biology, John Wiley & Sons; 5th edition (2002), ISBN: 0471250929).

In a further embodiment, the recombinant nucleic acid molecules according to the invention comprise a nucleic acid molecule according to the invention which is linked with regulatory sequences, which initiate transcription in prokaryotic or eukaryotic cells.

“Regulatory sequences, which initiate transcription” in a cell are also known as promoters.

Information concerning regulatory sequences and plasmids are well known to a person skilled in the art and are described e.g. by the Registry of Standard Biological Parts supported by The International Genetically Engineered Machine (iGEM) Foundation (One Kendall Square, Suite B6104, Cambridge, MA 02139, USA) in the world wide web (http://parts.igem.org/Catalog).

Regulatory sequences which initiate transcription in prokaryotic organisms, e.g. E. coli, and in eukaryotic organisms are sufficiently described in literature, in particular such for expression in yeast are described, e.g. Saccharomyces cerevisiae. An overview of various systems for expression for proteins in various host organisms can be found, for example, in Methods in Enzymology 153 (1987), 383-516 and in Bitter et al. (Methods in Enzymology 153 (1987), 516-544) or in Gomes et al. (2016, Advances in Animal and Veterinary Sciences, 4 (4), 346) and Baghban et al. (2018, Current Pharmaceutical Biotechnology, 19 (6)). Common yeast promoters are pAOX1, pHIS4, pGAL, pScADH2 (Baghban et al., 2018, see above). Common bacterial promoters are T5, T7, rhamnose-inducible, arabinose-inducible, PhoA, artificial trc (trp-lac) promoter as described by Marschall et al. (2017, Appl Microbiol Biotechnol 101, 501-512) and Tegel et al. (2011, FEBS Journal 278, 729-739).

A further embodiment of recombinant nucleic acid molecules of the present invention are vectors or plasmids, which comprise the nucleic acid molecules according to the invention.

“Vectors” are commonly understood in the field of molecular biology and herein to represent a nucleic acid sequence or a vehicle comprising a nucleic acid sequence used to transfer genetic material (DNA or RNA) into a target cell. Vectors can be plasmids, e.g. T-DNA or binary vectors for generating transgenic plants, expression vectors for expression of nucleic acid sequences in a host cell, shuttle vectors which are eligible to propagate in different hosts, or vectors can be virus particles or bacteriophages having been modified to deliver foreign genetic material into a host.

“Plasmids” are commonly understood in the field of molecular biology and herein to represent an autonomously self-replicating, often circular DNA molecule which is when present in a host cell separated from the chromosomal DNA.

Nucleic acid molecules according to the invention, recombinant nucleic acid molecules according to the invention, vectors or plasmids according to the invention can be used for production of proteins according to the invention, e.g. by expressing the nucleic acid molecules according to the invention in host cells.

Another embodiment of the invention concerns hosts or host cells comprising or expressing a nucleic acid molecule according to invention or comprising proteins according to the invention or comprising a recombinant nucleic acid molecule according to the invention or comprising a vector according to the invention or comprising a plasmid according to the invention.

Nucleic acid molecules according to the invention encoding a protein having the activity of a lipase can be expressed in host cells for e.g. their multiplication or for production of proteins according to the invention. For expression in host cells, nucleic acid molecules according to the invention can be comprised on vectors or plasmids or they can be stably integrated into the genome of a respective host cell. The nucleic acid molecules according to the invention can also be comprised by vectors which support their introduction into host cells.

A further embodiment of the present invention concerns a host or host cell according to the invention comprising a nucleic acid molecule according to the invention or comprising a recombinant nucleic acid molecule according to the invention or comprising a vector according to the invention or comprising a plasmid according to the invention and, in each case comprising a protein according to the invention.

Another embodiment of the present invention concerns a host or host cell according to the invention comprising a nucleic acid molecule according to the invention or comprising a recombinant nucleic acid molecule according to the invention or comprising a vector according to the invention or comprising a plasmid according to the invention and, in each case expressing a protein according to the invention.

Another embodiment of the present invention concerns a host or host cell according to the invention comprising a nucleic acid molecule according to the invention or comprising a recombinant nucleic acid molecule according to the invention or comprising a vector according to the invention or comprising a plasmid according to the invention and, in each case expressing a protein, wherein the protein has the activity of a lipase.

“Expressing a nucleic acid molecule” shall be understood herein to mean that in case the nucleic acid molecule is RNA or mRNA the nucleic acid molecule is translated into a protein, preferably translated into a protein having the activity of a lipase or in case of the nucleic acid molecule is DNA or cDNA it is transcribed (and in case of genomic DNA containing introns is processed) into mRNA, preferably into a mRNA encoding a protein having the activity of a lipase and subsequently translated into a protein, preferably translated into a protein having the activity of a lipase.

Transcription of a given nucleic acid molecule in a host can be demonstrated by methods known to a person skilled in the art, for example, by detection of specific transcripts (mRNA) of foreign nucleic acid molecules by Northern blot analysis or RT-PCR.

Whether hosts or host cells comprise a given protein or comprise a protein which is derived from expressing a nucleic acid molecule can be determined by methods known to a person skilled in the art, for example, by immunological methods, such as Western blot analysis, ELISA (Enzyme Linked Immuno Sorbent Assay) or RIA (Radio Immune Assay). The person skilled in the art is familiar with methods for preparing antibodies which react specifically with a certain protein, i.e. which bind specifically to a certain protein (see, for example, Lottspeich and Zorbas (eds.), 1998, Bioanalytik, Spektrum akad, Verlag, Heidelberg, Berlin, ISBN 3-8274-0041-4). Some companies (Thermo Fisher Scientific, 168 Third Avenue, Waltham, MA USA 0245; GenScript, 60 Centennial Ave., Piscataway, NJ 08854, USA) offer the preparation of such antibodies as an order service.

Furthermore, a person skilled in the art can test if a host or host cell comprises a protein according to the invention by detecting (additional) activity of proteins having the activity of a lipase in a respective host cell. Preferably activity of proteins having additional activity of a lipase in a respective host cell is detected by comparing the activities of lipases of a host cell according to the invention with the respective activity of host cell not comprising a protein according to the invention.

Testing if a protein has the activity of a lipase can be done by methods known in the art.

Host or host cells according to the invention can be produced by a person skilled in the art by known methods for genetically modifying or transforming organisms.

A further subject of the present invention therefore is a host or host cell according to the invention, particularly a prokaryotic or eukaryotic host or host cell, which is genetically modified (or transformed) with a nucleic acid molecule according to the invention or with a recombinant nucleic acid molecule according to the invention or with a vector according to the invention or a plasmid according to the invention. Preferably the genetically modified (transformed) host or host cell according to the invention expresses a protein having the activity of a lipase, more preferably, the genetically modified (transformed) host or host cell according to the invention expresses a protein according to the invention.

“Genetically modified with a nucleic acid molecule” or “transformed with a nucleic acid molecule” shall be understood herein to mean that a nucleic acid molecule is or was introduced into a host or host cell by technical and/or non-naturally occurring means, preferably by technical methods in the field of molecular biology, biotechnology or genetic modification.

Descendants, offspring or progeny of hosts or host cells according to the invention are also an embodiment of the invention, preferably these descendants, offspring or progeny comprise a nucleic acid molecule according to the invention or comprise a recombinant nucleic acid molecule according to the invention or comprise a vector according to the invention or comprise a plasmid according to the invention or comprise a protein according to the invention, more preferably these descendants, offspring or progeny comprise a nucleic acid molecule according to the invention or comprise a recombinant nucleic acid molecule according to the invention or comprise a vector according to the invention or comprise a plasmid according to the invention and, in each case express a protein, wherein the protein has the activity of a lipase, even more preferably these descendants, offspring or progeny comprise a nucleic acid molecule according to the invention or comprise a recombinant nucleic acid molecule according to the invention or comprise a vector according to the invention or comprise a plasmid according to the invention and, in each case express a protein, wherein the protein has the activity of a lipase according to the invention.

The host or host cell according to the invention can be a host or host cell from any prokaryotic or eucaryotic organism. The hosts or host cells can be bacteria or bacteria cells (e.g. E. coli, bacteria of the genus Bacillus, in particular Bacillus subtilis, Agrobacterium, particularly Agrobacterium tumefaciens or Agrobacterium rhizogenes, Pseudomonas, particularly Pseudomonas fluorescens, Streptomyces spp, Rhodococcus spp, in particular Rhodococcus rhodochrous, Vibrio natrigens, Corynebacterium, particularly Corynebacterium glutamicum) or fungi or fungal cells (e.g. Agaricus, in particular Agaricus bisporus, Aspergillus, Trichoderma or yeasts, particularly S. cerevisiae, Pichia ssp. like P. pastoris), as well as plants or plant cells or they can be animals or animal cells.

Preferred host cells according to the invention are cells of microorganisms. Within the framework of the present patent application, this is understood to include all bacteria and all protists (e.g. fungi, particularly yeasts and algae), as they are defined in Schlegel “General Microbiology” (Georg Thieme Publishing House (1985), 1-2), for example.

In respect to microorganisms, the hosts or host cells according to the invention are preferably bacteria/bacteria cells or yeast/yeast cells, most preferably they are bacteria/bacteria cells. Concerning bacteria/bacteria cells, the hosts or host cells according to the invention are preferably Bacillus species/Bacillus species cells or Escherichia coli/Escherichia coli cells cells most preferably Escherichia coli/Escherichia coli cells.

Alternatively, Pseudomonas, particularly Pseudomonas fluorescens, Streptomyces spp, Rhodococcus spp, in particular Rhodococcus rhodochrous, Vibrio spp, particularly Vibrio natrigens, Corynebacterium, particularly Corynebacterium glutamicum or others can be hosts or host cells according to the invention.

A preferred embodiment of the invention concerns hosts or host cells according to the invention comprising a nucleic acid molecule according to the invention, wherein the nucleic acid molecule according to the invention is characterized in that the codons of said nucleic acid molecule are changed such that they are adapted to the frequency of use of the codons of the host or a host cell, respectively.

Host cells according to the invention can be used for production of proteins according to the invention. Proteins according to the invention can be used in methods for production of enantiomerically enriched or nearly enantiomerically pure secondary alcohols.

A second aspect of the present invention relates to a method for hydrolysing a racemic substrate of formula (II) in an enantiomerically selective manner to separate enantiomerically enriched or pure compounds of formula (I), comprising contacting the substrate with a variant protein having the activity of a lipase according to the first aspect of the present invention or with a lipase according to SEQ ID No. 1,

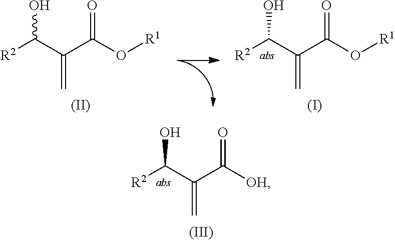

wherein R¹ and R² are independently from each other selected from a substituted or unsubstituted (n)-alkyl, iso-alkyl, unsubstituted aryl, alkyl-substituted or aryl-substituted aryl.

Preferably, the substrate is contacted with a variant protein having the activity of a lipase according to the first aspect of the present invention.

R¹ and R² are preferably independently from each other chosen to be a linear or branched C₁₋₁₀ residue.

Further preferably, R¹ is selected from methyl, iso-butyl, tert-butyl, and iso-propyl; and/or R² is methyl.

Even further preferably, R¹ is selected from methyl, iso-butyl, tert-butyl, and iso-propyl; and R² is methyl.

In a further embodiment of the invention, the method comprises hydrolysing a racemic substrate of formula (II-1) in an enantiomerically selective manner to separate an enantiomerically enriched or pure compound of formula (I-1),

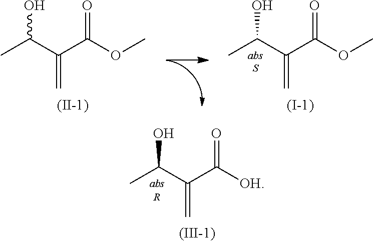

The method preferably comprises isolating the compound (I) or (I-1), and/or (III) or (III-1) after hydrolysis. The isolation of compounds (I-1) can be done by any means which are known to those skilled in the art and is preferably done via extraction and/or direct distillation.

The lipase variants or protein variants according to the invention show improved selectivity and/or improved specific activity regarding the stereoselective hydrolysis of methyl-3-hydroxy-2-methylene-butanoate (formula II-1) and are better adapted to isolate enantiomerically enriched or nearly pure substrate methyl (3S)-3-hydroxy-2-methylene-butanoate (formula I-1) compared to a wild-type lipase.

Compounds of formula (I)

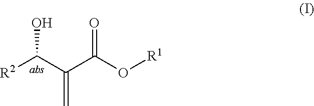

represent an important building block in the synthesis of complex agrochemical compounds. In particular, methyl (3S)-3-hydroxy-2-methylene-butanoate of formula (I-1)

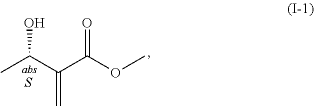

which is the S-enantiomer of racemic methyl 3-hydroxy-2-methylene-butanoate, is an important intermediate in the respective synthesis of agrochemical compounds described in WO 2018/228985. The lipase variants according to the present invention selectively hydrolyse the R-enantiomer of racemic methyl 3-hydroxy-2-methylene-butanoate, allowing the separation of the S-enantiomer of said racemic methyl 3-hydroxy-2-methylene-butanoate.

In a further embodiment of the invention, the method is conducted in an aqueous solution.

Preferably, the method is conducted in a mixture or biphasic liquid system of water and an organic solvent, such as methyl-tert-butylether, toluene, 2-methyltetrahydrofuran, methyl isobutyl ketone, cyclohexane, cyclopentyl methyl ether, chlorobenzene, tert-amylmethylether, ethyl acetate, isopropyl acetate. Even further preferably, the organic solvent is methyl-tert-butylether (MTBE).

The ratio of organic solvent to water is further preferably in the range from 1:1 to 6:1, even more preferably 2:1 to 4:1.

Surprisingly, it was found that a significant reduction of the aqueous phase, substituted partially by an organic solvent, not only allows for higher concentration of the final product, thus increasing the productivity, but also allows for the reduction of the enzyme/lipase load.

Furthermore, the presence of an organic solvent, such as MTBE, promotes an efficient downstream processing of the reaction products, i.e. isolation of compounds of the formula (I) or (I-1).

In a further embodiment of the invention, the method is carried out at a temperature of between 2° and 60° C., preferably between 3° and 55° C. Further preferably, the method is carried out at a temperature of between 35 and 50° C.

In a further embodiment of the invention, the method is carried out for at least 1 h, preferably at least 2 h, further preferably at least 3 h.

In a further embodiment of the invention, the method is carried out for at most 40 h, preferably at most 30 h, further preferably at most 20 h.

In a further embodiment of the invention, the method is carried out at a pH of between 7 and 8.5, preferably between 7.2 and 8.0, further preferably between 7.4 and 7.6. The pH may be adjusted by addition of organic or inorganic bases, preferably by the addition of inorganic bases, such as hydrogen carbonates or carbonates, hydrogen phosphates and hydroxides.

At a pH above 8.5 the enzyme is deactivated; further, there is partial hydrolysis of the ester of formula (I). Below pH 7, the reaction rate decreased and finally the reaction stops. Further below, at a pH of 4.5 or less, the enzyme is deactived.

The pH may be controlled by addition of a base, such as NaOH, KOH, K₂CO₃, KHCO₃, Na₂CO₃, NaHCO₃. Preferred bases are carbonates or hydrogencarbonates. The base can be added as solid or aqueous solution, diluted or saturated. The base may be added to the reaction before the substrate is dosed, or the base is dosed into the reaction in parallel to the substrate. The substrate and/or base can be dosed or added in one portion.

The lipase of SEQ ID No. 1 or the protein variant according to the first aspect of the present invention may be provided as purified enzyme or in form of spray-dried or freeze-dried biomass or as broth. Preferably, the protein variant is provided as broth, i.e. as cell culture, or the broth is centrifugated and the cell pellet is freeze-dried to obtain lyophilized cells or biomass.

While lyophilized biomass is easy to handle in terms of independent timing for performing the claimed method, the use of the broth/cell culture directly to perform the claimed method is cost and time efficient.

For downstream processing, the reaction is preferably conducted in a mixture of water and a water-immiscible solvent. In case of conducting the reaction in a biphasic mixture comprising water and an immiscible organic solvent, the phases are separated and the aqueous phase is eventually back-extracted.

The biomass can be separated by known technical means (e.g. centrifuge, filtration or decantation) before distillation or the distillation can take place without separation of the biomass.

It is to be understood in the context of the present invention that all embodiments concerning the method according to the second aspect can be combined with all different lipase variants according to the first aspect of the invention, and in particular with the preferred lipase variants. I.e. the method for hydrolysing a racemic substrate of formula (II) in an enantiomerically selective manner to separate enantiomerically enriched or pure compounds of formula (I) may be carried out by means of a protein having the activity of a lipase wherein the protein is encoded by an amino acid sequence having at least 80%, preferably 85%, more preferably 90%, furthermore preferably 92%, even more preferably 95% with the amino acid sequence shown under SEQ ID No. 1,
characterized in that the amino acid sequence of the variant differs from the amino acid sequence of SEQ ID NO. 1 in at least one of the following positions:
i. the amino acid at position 44 is different from L, preferably the amino acid at position 44 is M, W or Y;
ii. the amino acid at position 51 is different from F, preferably the amino acid at position 51 is N, or M;
iii. the amino acid at position 52 is different from V, preferably the amino acid at position 52 is L;
iv. the amino acid at position 53 is different from T, preferably the amino acid at position 53 is S, P, I, E, or A;
v. the amino acid at position 54 is different from D, preferably the amino acid at position 54 is Q, M, F, G, E, L, T or P;
vi. the amino acid at position 55 is different from A, preferably the amino acid at position 55 is R, M, D, Y, S, or I;
vii. the amino acid at position 109 is different from G, preferably the amino acid at position 109 is H or F;
viii. the amino acid at position 110 is different from M, preferably the amino acid at position 110 is T or V;
ix. the amino acid at position 111 is different from A, preferably the amino acid at position 111 is T or S;
X. the amino acid at position 117 is different from Y, preferably the amino acid at position 117 is F or S;
xi. the amino acid at position 121 is different from Y, preferably the amino acid at position 121 is V;
xii. the amino acid at position 122 is different from K, preferably the amino acid at position 122 is Q, A, Y, R, or V;
xiii. the amino acid at position 153 is different from H, preferably the amino acid at position 153 is N, Y, D, E, or C;
xiv, the amino acid at position 160 is different from T, preferably the amino acid at position 160 is E, C, D, P, I, Q, K, M, S, F, A, or N;
xv. the amino acid at position 179 is different from D, preferably the amino acid at position 179 is C;
xvi. the amino acid at position 181 is different from A, preferably the amino acid at position 181 is Q;
xvii. the amino acid at position 184 is different from A, preferably the amino acid at position 184 is G or T;
xviii. the amino acid at position 211 is different from Y, preferably the amino acid at position 211 is E;
xix. the amino acid at position 212 is different from A, preferably the amino acid at position 212 is S or P;
xx. the amino acid at position 216 is different from Y, preferably the amino acid at position 216 is K or A;
xxi. the amino acid at position 234 is different from S, preferably the amino acid at position 234 is K, T or G;
xxii. the amino acid at position 235 is different from S, preferably the amino acid at position 235 is V or M;
xxiii. the amino acid at position 236 is different from K, preferably the amino acid at position 236 is T;
xxiv. the amino acid at position 238 is different from R, preferably the amino acid at position 238 is A, K, D, E, or Q;
xxv. the amino acid at position 240 is different from Y, preferably the amino acid at position 240 is F;
xxvi. the amino acid at position 289 is different from D, preferably the amino acid at position 289 is S or G;
xxvii. the amino acid at position 291 is different from G, preferably the amino acid at position 291 is E or W;
xxviii. the amino acid at position 317 is different from N, preferably the amino acid at position 317 is T;
xxix. the amino acid at position 320 is different from N, preferably the amino acid at position 320 is E or G;
xxx. the amino acid at position 321 is different from L, preferably the amino acid at position 321 is F.

The amino acid sequence of the variant protein lipase used in the method for hydrolysing a racemic substrate of formula (II) in an enantiomerically selective manner to separate enantiomerically enriched or pure compounds of formula (I) may differ from the amino acid sequence of SEQ ID NO. 1 by at least one of the following modifications:
i. the amino acid at position 44 is W or Y;
ii. the amino acid at position 54 is F;
iii. the amino acid at position 55 is R;
iv. the amino acid at position 109 is H;
v. the amino acid at position 110 is T or V;
vi. the amino acid at position 117 is F;
vii. the amino acid at position 122 is Q or R;
viii. the amino acid at position 160 is E;
ix. the amino acid at position 216 is K;
x. the amino acid at position 236 is T;
xi. the amino acid at position 238 is K or E;
xii. the amino acid at position 240 is F.

Preferably, the amino acid sequence of the variant protein lipase used in the method for hydrolysing a racemic substrate of formula (II) in an enantiomerically selective manner to separate enantiomerically enriched or pure compounds of formula (I) differs from the amino acid sequence of SEQ ID NO. 1 by at least the mutation G109H, i.e. the amino acid at position 109 is H.

The amino acid sequence of the variant protein lipase used in the method for hydrolysing a racemic substrate of formula (II) in an enantiomerically selective manner to separate enantiomerically enriched or pure compounds of formula (I) may differ from the amino acid sequence of SEQ ID NO. 1 by at least two, further preferably at least three, even further preferably at least four, in particular preferably at least five of the following modifications selected from:
i. the amino acid at position 57 is different from N, preferably the amino acid is P;
ii. the amino acid at position 109 is different from G, preferably the amino acid is H;
iii. the amino acid at position 122 is different from K, preferably the amino acid is R;
iv. the amino acid at position 212 is different from A, preferably the amino acid is P;
V. the amino acid at position 234 is different from S, preferably the amino acid is K;
vi. the amino acid at position 289 is different from D, preferably the amino acid is G.

Preferably, the amino acid sequence of the variant differs from the amino acid sequence of SEQ ID NO. 1 by the following modifications:
i. the amino acid at position 57 is P;
ii. the amino acid at position 109 is H;
iii. the amino acid at position 212 is P;
iv. the amino acid at position 234 is K; and
V. the amino acid at position 289 is G.

Further preferably, the amino acid sequence of the variant differs from the amino acid sequence of SEQ ID NO. 1 by the following modifications:
i. the amino acid at position 57 is P;
ii. the amino acid at position 109 is H;
iii. the amino acid at position 122 is R;
iv. the amino acid at position 212 is P;
v. the amino acid at position 234 is K; and
vi. the amino acid at position 289 is G.

The amino acid sequence of the variant protein lipase used in the method for hydrolysing a racemic substrate of formula (II) in an enantiomerically selective manner to separate enantiomerically enriched or pure compounds of formula (I) may differ from the amino acid sequence of SEQ ID NO. 1 by the following modifications:
the amino acid at position 57 is P; the amino acid at position 109 is H; the amino acid at position 122 is R or K or Q, preferably R; the amino acid at position 212 is P; the amino acid at position 234 is K; the amino acid at position 289 is G;
and additionally at least one of the following modifications: the amino acid at position 44 is W or Y; the amino acid at position 54 is F, the amino acid at position 55 is R, the amino acid at position 110 is V or T; the amino acid at position 111 is T, the amino acid at position 117 is F, the amino acid at position 160 is E, the amino acid at position 216 is K, the amino acid at position 236 is T, the amino acid at position 238 is K or E, the amino acid at position 240 is F.

Preferred proteins according to the invention encoding lipases that may be used in the method for hydrolysing a racemic substrate of formula (II) in an enantiomerically selective manner to separate enantiomerically enriched or pure compounds of formula (I) are shown under SEQ ID Nos. 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239.

A further embodiment of the invention is the use of a protein according to the first aspect of the invention for the stereoselective hydrolysis of racemic methyl-3-hydroxy-2-methylene-butanoate to isolate methyl (3S)-3-hydroxy-2-methylene-butanoate.

“Enantiomerically enriched” means herein that one of two enantiomers is present in a composition in higher amounts than the other enantiomer, preferably at least 60% of one enantiomer is present in the composition, more preferably at least 65% of one enantiomer is present in the composition, further more preferably at least 70% of one enantiomer is present in the composition, even more preferably at least 75% of one enantiomer is present in the composition, even further more preferably at least 80% of one enantiomer is present in the composition, particular preferably at least 85% of one enantiomer is present in the composition, most preferably at least 90% of one enantiomer is present in the composition or especially preferably at least 94% of one enantiomer is present in the composition.

“Enantiomerically nearly pure” means herein that one of two enantiomers is present in a composition in amounts of at least 95.0%, preferably one of two enantiomers is present in a composition in amounts of at least 95.5%, more preferably one of two enantiomers is present in a composition in amounts of at least 96.0%, further more preferably one of two enantiomers is present in a composition in amounts of at least 96.5%, even more preferably one of two enantiomers is present in a composition in amounts of at least 97.0%, even further more preferably one of two enantiomers is present in a composition in amounts of at least 98.0%, particular preferably one of two enantiomers is present in a composition in amounts of at least 98.5%, most preferably one of two enantiomers is present in a composition in amounts of at least 99.0%, or especially preferably one of two enantiomers is present in a composition in amounts of at least 99.5%.

A third aspect of the present invention relates to a method for the enantiomeric enrichment of compounds of formula (I)

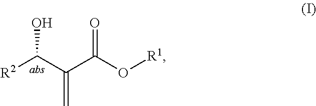

comprising reacting compounds of formula (II) with compounds of formula (IV)

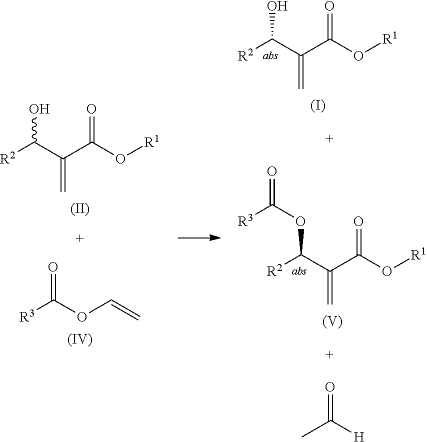

and in particular

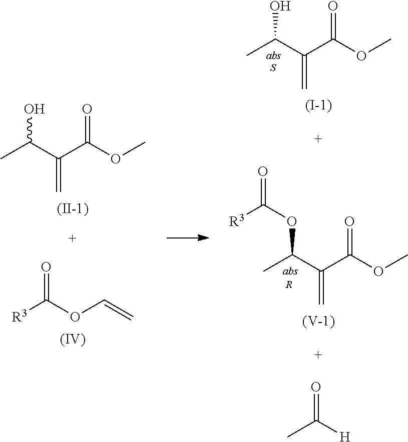

with R¹ and R² being defined as above, and R³ being a substituted or unsubstituted (n)-Alkyl or iso-alkyl,
in the presence of a lipase under anhydrous conditions.

“Anhydrous conditions” refer to a reaction system comprising at most 3% water in the liquid phase.

The reaction preferably takes place in an organic solvent, such as hexane or n-heptane, or without any additional solvent.

R³ can preferably be a linear C₁₋₁₆ residue, in particular methyl or unsubstituted and saturated C₈-C₁₂ residue. Compound (IV) can be in particular vinyl acetate or vinyl laurate.

The lipase is preferably immobilized on a solid carrier during the action. Immobilization leads to increased enzyme stability in non-aqueous solutions.

The reaction is preferably carried out at a temperature of between 2° and 40° C., preferably 25 to 35° C. Such temperature range allows for optimal enzymatic reacticity.

The reaction is preferably carried out for at least 1 h, further preferably at least 3 h, even further preferably at least 5 h.

The lipase is preferably recycled after the reaction has taken place. This allows the method to be designed in a cost-efficient manner. The lipase can thus be used multiple times for the reaction according to the third aspect or a further reaction.

The compound of formula (I) is preferably isolated by means of distillation directly from the reaction mixture or from the supernatant, obtained after decanting the reaction mixture. The lipase preferably remains in the remaining reaction mixture and can be reused for another reaction.

The lipase is preferably CALB lipase. CALB is a non-specific lipase originating from Candida antarctica B and was first described in 1994 (Uppenberg J, Patkar S, Bergfors T, Jones TA (1994) J Mol Biol 235 (2): 790-792).

The lipase is furthermore preferably immobilized on a hydrophobic carrier, such as acrylic resin. A commercial version of CALB is, for example, Novozym® 435, which can be used for carrying out the reaction.

### DETAILED DESCRIPTION

The polypeptides (enzymes, i.e. lipases) as well as the processes according to the present invention allow for an efficient enantiomeric enrichment of compounds of the formula (I)

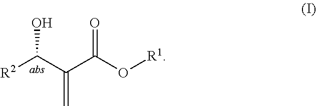

It was found that introduction of certain amino acid modifications into protein variants according to the invention improves the activity of the lipase, in particular in respect to its substrate specificity, meaning that these further modified lipase variants are better adapted to produce enantiomerically enriched or nearly pure products compared to known lipase variants. This in particular refers to the substrate (I-1). Enantionerically pure or at least enriched methyl (3S)-3-hydroxy-2-methylene-butanoate

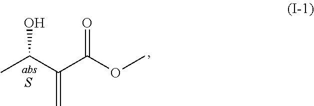

which is the S-enantiomer of racemic methyl 3-hydroxy-2-methylene-butanoate, is an important intermediate in the respective synthesis of the compounds described in WO 2018/228985. The lipase variants of the present invention catalyse entioselectively the hydrolysis of the racemic methyl 3-hydroxy-2-methylene-butanoate into (3R)-3-hydroxy-2-methylene-butanoic acid, leaving methyl (3S)-3-hydroxy-2-methylene-butanoate unhydrolyzed.

The terms used herein are known to those skilled in the art. Otherwise, the following definitions are used:

For the purposes of the present invention, the term “alkyl” includes saturated hydrocarbon residues which can be branched or straight-chain and unsubstituted or at least monosubstituted. Examples of suitable alkyl residues, which can be unsubstituted or mono- or polysubstituted, are methyl, ethyl, n-propyl, isopropyl, n-butyl, isobutyl, 2-butyl, tert-butyl, n-pentyl, 2-pentyl, 3-pentyl, isopentyl, neo-pentyl, n-hexyl, 2-hexyl, 3-hexyl, n-heptyl, n-octyl, —C(H)(C2H5)2, —C(H)(n-C3H7) 2 and —CH2-CH2-C(H)(CH3)-(CH2) 3-CH3.

Unless defined otherwise elsewhere, the term “aryl” either alone or in combination with other terms, is to be understood as a mono- or polycyclic, preferably a mono- or bicyclic, aromatic hydrocarbon radical with preferably 6, 10 or 14 carbon atoms. An aryl radical can be unsubstituted or monosubstituted or polysubstituted, either with identical or different substituents.

### Description of the Sequences

Throughout the application, nucleotide and amino acid abbreviations are used according to the following IUPAC codes:

TABLE 1

| IUPAC | |
|---|---|
| nucleotide | |
| code | Base |
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| — | gap |

For discrimination between amino acids and nucleotides, capitalized nucleotide code abbreviations given in above Table are written herein in lower case.

TABLE 2

| IUPAC | | |
|---|---|---|
| amino | Three | |
| acid | letter | |
| code | code | Amino acid |
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Codon usage follows herein the so called “general genetic code” according to the following Table, wherein “t” is to be substituted by “u” in ribonucleic acid (RNA) sequences.

TABLE 3

| | Three | Single | | Codons due to |
|---|---|---|---|---|
| | letter | letter | DNA | degenerate |
| Amino Acid | code | code | codons | genetic code |
| Alanine | Ala | A | gca | gcn |
| Alanine | Ala | A | gcc | gcn |
| Alanine | Ala | A | gcg | gcn |
| Alanine | Ala | A | gct | gcn |
| Arginine | Arg | R | aga | mgn |
| Arginine | Arg | R | agg | mgn |
| Arginine | Arg | R | cga | mgn |
| Arginine | Arg | R | cgc | mgn |
| Arginine | Arg | R | cgg | mgn |
| Arginine | Arg | R | cgt | mgn |
| Asparagine | Asn | N | aac | aay |
| Asparagine | Asn | N | aat | aay |
| Aspartic acid | Asp | D | gac | gay |
| Aspartic acid | Asp | D | gat | gay |
| Cysteine | Cys | C | tgc | tgy |
| Cysteine | Cys | C | tgt | tgy |
| Glutamic acid | Glu | E | gaa | gar |
| Glutamic acid | Glu | E | gag | gar |
| Glutamine | Gln | Q | caa | car |
| Glutamine | Gln | Q | cag | car |
| Glycine | Gly | G | gga | ggn |
| Glycine | Gly | G | ggc | ggn |
| Glycine | Gly | G | ggg | ggn |
| Glycine | Gly | G | ggt | ggn |
| Histidine | His | H | cac | cay |
| Histidine | His | H | cat | cay |
| Isoleucine | Ile | I | ata | ath |
| Isoleucine | Ile | I | atc | ath |
| Isoleucine | Ile | I | att | ath |
| Leucine | Leu | L | cta | ytn |
| Leucine | Leu | L | ctc | ytn |
| Leucine | Leu | L | ctg | ytn |
| Leucine | Leu | L | ctt | ytn |
| Leucine | Leu | L | tta | ytn |
| Leucine | Leu | L | ttg | ytn |
| Lysine | Lys | K | aaa | aar |
| Lysine | Lys | K | aag | aar |
| Methionine | Met | M | atg | atg |
| Phenylalanine | Phe | F | ttc | tty |
| Phenylalanine | Phe | F | ttt | tty |
| Proline | Pro | P | cca | ccn |
| Proline | Pro | P | ccc | ccn |
| Proline | Pro | P | ccg | ccn |
| Proline | Pro | P | cct | ccn |
| Serine | Ser | S | agc | wsn |
| Serine | Ser | S | agt | wsn |
| Serine | Ser | S | tca | wsn |
| Serine | Ser | S | tcc | wsn |
| Serine | Ser | S | tcg | wsn |
| Serine | Ser | S | tct | wsn |
| Threonine | Thr | T | aca | acn |
| Threonine | Thr | T | acc | acn |
| Threonine | Thr | T | acg | acn |
| Threonine | Thr | T | act | acn |
| Tryptophan | Thr | W | tgg | tgg |
| Tyrosine | Tyr | Y | tac | tay |
| Tyrosine | Tyr | Y | tat | tay |
| Valine | Val | V | gta | gtn |
| Valine | Val | V | gtc | gtn |
| Valine | Val | V | gtg | gtn |
| Valine | Val | V | gtt | gtn |
| Stop codons | Stop | Stop | taa | trr |
| Stop codons | Stop | Stop | tag | trr |
| Stop codons | Stop | Stop | tga | trr |

The sequence listing associated with this application is filed in electronic format and hereby incorporated by reference into the specification in its entirety. “PRT” stands for “protein” and “NUC” for “nucleic acid”.

TABLE 4a
Lipase variants according to the first aspect of the present invention
(SEQ ID No. 7-169) and further lipases (SEQ ID No. 1-6)

| SEQ | | | |
|---|---|---|---|
| ID | | Mutations/Variations | |
| No | Subject | with respect to SEQ ID No. 1 | Type |
| 1 | Lipase | — | PRT |
| 2 | Lipase | — | NUC |
| 3 | Reference lipase variant | Q295V, K298A | PRT |
| 4 | Reference lipase variant | Q295V, K298A | NUC |
| 5 | Reference lipase variant | V33I, I254S | PRT |
| 6 | Reference lipase variant | V33I, I254S | NUC |
| 7 | Lipase variant | G109H | PRT |
| 8 | Lipase variant | G109H | NUC |
| 9 | Lipase variant | L44M | PRT |
| 10 | Lipase variant | L44M | NUC |
| 11 | Lipase variant | L44W | PRT |
| 12 | Lipase variant | L44W | NUC |
| 13 | Lipase variant | K122Q | PRT |
| 14 | Lipase variant | K122Q | NUC |
| 15 | Lipase variant | L44Y | PRT |
| 16 | Lipase variant | L44Y | NUC |
| 17 | Lipase variant | A184G | PRT |
| 18 | Lipase variant | A184G | NUC |
| 19 | Lipase variant | A212S | PRT |
| 20 | Lipase variant | A212S | NUC |
| 21 | Lipase variant | Y117F | PRT |
| 22 | Lipase variant | Y117F | NUC |
| 23 | Lipase variant | Y216K | PRT |
| 24 | Lipase variant | Y216K | NUC |
| 25 | Lipase variant | Y117S | PRT |
| 26 | Lipase variant | Y117S | NUC |
| 27 | Lipase variant | M110T | PRT |
| 28 | Lipase variant | M110T | NUC |
| 29 | Lipase variant | T160E | PRT |
| 30 | Lipase variant | T160E | NUC |
| 31 | Lipase variant | L321F | PRT |
| 32 | Lipase variant | L321F | NUC |
| 33 | Lipase variant | M110V | PRT |
| 34 | Lipase variant | M110V | NUC |
| 35 | Lipase variant | A181Q | PRT |
| 36 | Lipase variant | A181Q | NUC |
| 37 | Lipase variant | T160C | PRT |
| 38 | Lipase variant | T160C | NUC |
| 39 | Lipase variant | Y240F | PRT |
| 40 | Lipase variant | Y240F | NUC |
| 41 | Lipase variant | T160D | PRT |
| 42 | Lipase variant | T160D | NUC |
| 43 | Lipase variant | T160P | PRT |
| 44 | Lipase variant | T160P | NUC |
| 45 | Lipase variant | T160I | PRT |
| 46 | Lipase variant | T160I | NUC |
| 47 | Lipase variant | T160Q | PRT |
| 48 | Lipase variant | T160Q | NUC |
| 49 | Lipase variant | T160K | PRT |
| 50 | Lipase variant | T160K | NUC |
| 51 | Lipase variant | T160M | PRT |
| 52 | Lipase variant | T160M | NUC |
| 53 | Lipase variant | D54Q | PRT |
| 54 | Lipase variant | D54Q | NUC |
| 55 | Lipase variant | D289S | PRT |
| 56 | Lipase variant | D289S | NUC |
| 57 | Lipase variant | D54M | PRT |
| 58 | Lipase variant | D54M | NUC |
| 59 | Lipase variant | T53S | PRT |
| 60 | Lipase variant | T53S | NUC |
| 61 | Lipase variant | R238A | PRT |
| 62 | Lipase variant | R238A | NUC |
| 63 | Lipase variant | R238K | PRT |
| 64 | Lipase variant | R238K | NUC |
| 65 | Lipase variant | N320E | PRT |
| 66 | Lipase variant | N320E | NUC |
| 67 | Lipase variant | H153N | PRT |
| 68 | Lipase variant | H153N | NUC |
| 69 | Lipase variant | D54F | PRT |
| 70 | Lipase variant | D54I | NUC |
| 71 | Lipase variant | K122A | PRT |
| 72 | Lipase variant | K122A | NUC |
| 73 | Lipase variant | R238D | PRT |
| 74 | Lipase variant | R238D | NUC |
| 75 | Lipase variant | D54G | PRT |
| 76 | Lipase variant | D54G | NUC |
| 77 | Lipase variant | N317T | PRT |
| 78 | Lipase variant | N317T | NUC |
| 79 | Lipase variant | T160S | PRT |
| 80 | Lipase variant | T160S | NUC |
| 81 | Lipase variant | R238E | PRT |
| 82 | Lipase variant | R238E | NUC |
| 83 | Lipase variant | T53P | PRT |
| 84 | Lipase variant | T53P | NUC |
| 85 | Lipase variant | R238Q | PRT |
| 86 | Lipase variant | R238Q | NUC |
| 87 | Lipase variant | Y216A | PRT |
| 88 | Lipase variant | Y216A | NUC |
| 89 | Lipase variant | S234K | PRT |
| 90 | Lipase variant | S234K | NUC |
| 91 | Lipase variant | T160F | PRT |
| 92 | Lipase variant | T160F | NUC |
| 93 | Lipase variant | H153Y | PRT |
| 94 | Lipase variant | H153Y | NUC |
| 95 | Lipase variant | D54E | PRT |
| 96 | Lipase variant | D54E | NUC |
| 97 | Lipase variant | G291E | PRT |
| 98 | Lipase variant | G291E | NUC |
| 99 | Lipase variant | T53I | PRT |
| 100 | Lipase variant | T53I | NUC |
| 101 | Lipase variant | G291A | PRT |
| 102 | Lipase variant | G291A | NUC |
| 103 | Lipase variant | D54L | PRT |
| 104 | Lipase variant | D54L | NUC |
| 105 | Lipase variant | T160A | PRT |
| 106 | Lipase variant | T160A | NUC |
| 107 | Lipase variant | D54T | PRT |
| 108 | Lipase variant | D54T | NUC |
| 109 | Lipase variant | A55R | PRT |
| 110 | Lipase variant | A55R | NUC |
| 111 | Lipase variant | H153D | PRT |
| 112 | Lipase variant | H153D | NUC |
| 113 | Lipase variant | T53E | PRT |
| 114 | Lipase variant | T53E | NUC |
| 115 | Lipase variant | G109F | PRT |
| 116 | Lipase variant | G109F | NUC |
| 117 | Lipase variant | A55M | PRT |
| 118 | Lipase variant | A55M | NUC |
| 119 | Lipase variant | T53A | PRT |
| 120 | Lipase variant | T53A | NUC |
| 121 | Lipase variant | T160N | PRT |
| 122 | Lipase variant | T160N | NUC |
| 123 | Lipase variant | N320G | PRT |
| 124 | Lipase variant | N320G | NUC |
| 125 | Lipase variant | F51N | PRT |
| 126 | Lipase variant | F51N | NUC |
| 127 | Lipase variant | F51M | PRT |
| 128 | Lipase variant | F51M | NUC |
| 129 | Lipase variant | D179C | PRT |
| 130 | Lipase variant | D179C | NUC |
| 131 | Lipase variant | D289G | PRT |
| 132 | Lipase variant | D289G | NUC |
| 133 | Lipase variant | A55D | PRT |
| 134 | Lipase variant | A55D | NUC |
| 135 | Lipase variant | A111S | PRT |
| 136 | Lipase variant | A111S | NUC |
| 137 | Lipase variant | A55Y | PRT |
| 138 | Lipase variant | A55Y | NUC |
| 139 | Lipase variant | V52L | PRT |
| 140 | Lipase variant | V52L | NUC |
| 141 | Lipase variant | K236T | PRT |
| 142 | Lipase variant | K236T | NUC |
| 143 | Lipase variant | H153E | PRT |
| 144 | Lipase variant | H153E | NUC |
| 145 | Lipase variant | S234T | PRT |
| 146 | Lipase variant | S234T | NUC |
| 147 | Lipase variant | K122Y | PRT |
| 148 | Lipase variant | K122Y | NUC |
| 149 | Lipase variant | K122R | PRT |
| 150 | Lipase variant | K122R | NUC |
| 151 | Lipase variant | Y121V | PRT |
| 152 | Lipase variant | Y121V | NUC |
| 153 | Lipase variant | A184T | PRT |
| 154 | Lipase variant | A184T | NUC |
| 155 | Lipase variant | A55S | PRT |
| 156 | Lipase variant | A55S | NUC |
| 157 | Lipase variant | H153C | PRT |
| 158 | Lipase variant | H153C | NUC |
| 159 | Lipase variant | D54P | PRT |
| 160 | Lipase variant | D54P | NUC |
| 161 | Lipase variant | K122V | PRT |
| 162 | Lipase variant | K122V | NUC |
| 163 | Lipase variant | Y211E | PRT |
| 164 | Lipase variant | Y211E | NUC |
| 165 | Lipase variant | S234G | PRT |
| 166 | Lipase variant | S234G | NUC |
| 167 | Lipase variant | A55I | PRT |
| 168 | Lipase variant | A55I | NUC |
| 169 | Lipase variant | N57P, K122R, A212P, S234K, D289G | PRT |
| 170 | Lipase variant | N57P, K122R, A212P, S234K, D289G | NUC |
| 241 | Reference lipase variant | T188S, I254S, P302L, Q304E | PRT |
| 242 | Reference lipase variant | T188S, I254S, P302L, Q304E | NUC |

TABLE 4b
Further lipase variants according to the present invention (protein SEQ ID No. 171-239). The variants described
contain the backbone mutations N57P, K122R, A212P, S234K, D289G and G109H (SEQ ID No. 171, 172),
unless (i) the mutation “R122K” is indicated in addition, which means that the K122R mutation has been back-
mutated such that the position 122 carries an K again, or (ii) the mutation “R122Q” is indicated in addition,
which means that the K122R mutation has been further mutated such that the position 122 carries a Q.

| SEQ ID No | Subject | Mutations/Variations | | Type |
|---|---|---|---|---|
| 171 | Lipase variant | N57P, K122R, A212P, | | PRT |
| | | S234K, D289G, G109H, | | |
| 172 | Lipase variant | N57P, K122R, A212P, | | NUC |
| | | S234K, D289G, G109H | | |
| 173 | Lipase variant | N57P, K122R, A212P, | A55R, | PRT |
| | | S234K, D289G, G109H, | K236T, | |
| | | | R238K | |
| 174 | Lipase variant | N57P, K122R, A212P, | A55R, | NUC |
| | | S234K, D289G, G109H, | K236T, | |
| | | | R238K | |
| 175 | Lipase variant | N57P, K122R, A212P, | L44Y, | PRT |
| | | S234K, D289G, G109H, | T160E, | |
| | | | K236T | |
| 176 | Lipase variant | N57P, K122R, A212P, | L44Y, | NUC |
| | | S234K, D289G, G109H, | T160E, | |
| | | | K236T | |
| 177 | Lipase variant | N57P, K122R, A212P, | L44W, | PRT |
| | | S234K, D289G, G109H, | D54F, | |
| | | | T160E, | |
| | | | K236T, | |
| | | | R238E | |
| 178 | Lipase variant | N57P, K122R, A212P, | L44W, | NUC |
| | | S234K, D289G, G109H, | D54F, | |
| | | | T160E, | |
| | | | K236T, | |
| | | | R238E | |
| 179 | Lipase variant | N57P, K122R, A212P, | L44Y, | PRT |
| | | S234K, D289G, G109H, | T160E, | |
| | | | K236T, | |
| | | | Y240F | |
| 180 | Lipase variant | N57P, K122R, A212P, | L44Y, | NUC |
| | | S234K, D289G, G109H, | T160E, | |
| | | | K236T, | |
| | | | Y240F | |
| 181 | Lipase variant | N57P, K122R, A212P, | M110T, | PRT |
| | | S234K, D289G, G109H, | K236T, | |
| | | | Y240F | |
| 182 | Lipase variant | N57P, K122R, A212P, | M110T, | NUC |
| | | S234K, D289G, G109H, | K236T, | |
| | | | Y240F | |
| 183 | Lipase variant | N57P, K122R, A212P, | T160E, | PRT |
| | | S234K, D289G, G109H, | Y240F | |
| 184 | Lipase variant | N57P, K122R, A212P, | T160E, | NUC |
| | | S234K, D289G, G109H, | Y240F | |
| 185 | Lipase variant | N57P, K122R, A212P, | L44Y, | PRT |
| | | S234K, D289G, G109H, | K236T, | |
| | | | R238K | |
| 186 | Lipase variant | N57P, K122R, A212P, | L44Y, | NUC |
| | | S234K, D289G, G109H, | K236T, | |
| | | | R238K | |
| 187 | Lipase variant | N57P, K122R, A212P, | LA4Y, A55R, | PRT |
| | | S234K, D289G, G109H, | K236T | |
| 188 | Lipase variant | N57P, K122R, A212P, | LA4Y, A55R, | NUC |
| | | S234K, D289G, G109H, | K236T | |
| 189 | Lipase variant | N57P, K122R, A212P, | LA4Y, | PRT |
| | | S234K, D289G, G109H, | R238E | |
| 190 | Lipase variant | N57P, K122R, A212P, | LA4Y, | NUC |
| | | S234K, D289G, G109H, | R238E | |
| 191 | Lipase variant | N57P, K122R, A212P, | Y240F | PRT |
| | | S234K, D289G, G109H, | | |
| 192 | Lipase variant | N57P, K122R, A212P, | Y240F | NUC |
| | | S234K, D289G, G109H, | | |
| 193 | Lipase variant | N57P, K122R, A212P, | Y117F, | PRT |
| | | S234K, D289G, G109H, | R122K, | |
| | | | K236T, | |
| | | | R238K | |
| 194 | Lipase variant | N57P, K122R, A212P, | Y117F, | NUC |
| | | S234K, D289G, G109H, | R122K, | |
| | | | K236T, | |
| | | | R238K | |
| 195 | Lipase variant | N57P, K122R, A212P, | R122Q, | PRT |
| | | S234K, D289G, G109H, | T160E, | |
| | | | Y240F | |
| 196 | Lipase variant | N57P, K122R, A212P, | R122Q, | NUC |
| | | S234K, D289G, G109H, | T160E, | |
| | | | Y240F | |
| 197 | Lipase variant | N57P, K122R, A212P, | M110V, | PRT |
| | | S234K, D289G, G109H, | K236T, | |
| | | | R238E | |
| 198 | Lipase variant | N57P, K122R, A212P, | M110V, | NUC |
| | | S234K, D289G, G109H, | K236T, | |
| | | | R238E | |
| 199 | Lipase variant | N57P, K122R, A212P, | M110V | PRT |
| | | S234K, D289G, G109H, | | |
| 200 | Lipase variant | N57P, K122R, A212P, | M110V | NUC |
| | | S234K, D289G, G109H, | | |
| 201 | Lipase variant | N57P, K122R, A212P, | T160E | PRT |
| | | S234K, D289G, G109H, | | |
| 202 | Lipase variant | N57P, K122R, A212P, | T160E | NUC |
| | | S234K, D289G, G109H, | | |
| 203 | Lipase variant | N57P, K122R, A212P, | R238E | PRT |
| | | S234K, D289G, G109H, | | |
| 204 | Lipase variant | N57P, K122R, A212P, | R238E | NUC |
| | | S234K, D289G, G109H, | | |
| 205 | Lipase variant | N57P, K122R, A212P, | D54F, | PRT |
| | | S234K, D289G, G109H, | M110V, | |
| | | | T160E | |
| 206 | Lipase variant | N57P, K122R, A212P, | D54F, | NUC |
| | | S234K, D289G, G109H, | M110V, | |
| | | | T160E | |
| 207 | Lipase variant | N57P, K122R, A212P, | L44W, | PRT |
| | | S234K, D289G, G109H, | M110T, | |
| | | | A111T, | |
| | | | R122Q | |
| 208 | Lipase variant | N57P, K122R, A212P, | L44W, | NUC |
| | | S234K, D289G, G109H, | M110T, | |
| | | | A111T, | |
| | | | R122Q | |
| 209 | Lipase variant | N57P, K122R, A212P, | R122K, | PRT |
| | | S234K, D289G, G109H, | T160E | |
| 210 | Lipase variant | N57P, K122R, A212P, | R122K, | NUC |
| | | S234K, D289G, G109H, | T160E | |
| 211 | Lipase variant | N57P, K122R, A212P, | T160E, | PRT |
| | | S234K, D289G, G109H, | R238E | |
| 212 | Lipase variant | N57P, K122R, A212P, | T160E, | NUC |
| | | S234K, D289G, G109H, | R238E | |
| 213 | Lipase variant | N57P, K122R, A212P, | Y117F, | PRT |
| | | S234K, D289G, G109H, | R122Q, | |
| | | | K236T, | |
| | | | R238E | |
| 214 | Lipase variant | N57P, K122R, A212P, | Y117F, | NUC |
| | | S234K, D289G, G109H, | R122Q, | |
| | | | K236T, | |
| | | | R238E | |
| 215 | Lipase variant | N57P, K122R, A212P, | M110T | PRT |
| | | S234K, D289G, G109H, | | |
| 216 | Lipase variant | N57P, K122R, A212P, | M110T | NUC |
| | | S234K, D289G, G109H, | | |
| 217 | Lipase variant | N57P, K122R, A212P, | R122K, | PRT |
| | | S234K, D289G, G109H, | R238E | |
| 218 | Lipase variant | N57P, K122R, A212P, | R122K, | NUC |
| | | S234K, D289G, G109H, | R238E | |
| 219 | Lipase variant | N57P, K122R, A212P, | D54F, | PRT |
| | | S234K, D289G, G109H, | Y117F, | |
| | | | R122K, | |
| | | | T160E | |
| 220 | Lipase variant | N57P, K122R, A212P, | D54F, | NUC |
| | | S234K, D289G, G109H, | Y117F, | |
| | | | R122K, | |
| | | | T160E | |
| 221 | Lipase variant | N57P, K122R, A212P, | M110T, | PRT |
| | | S234K, D289G, G109H, | A111T | |
| 222 | Lipase variant | N57P, K122R, A212P, | M110T, | NUC |
| | | S234K, D289G, G109H, | A111T | |
| 223 | Lipase variant | N57P, K122R, A212P, | M110V, | PRT |
| | | S234K, D289G, G109H, | Y117F, | |
| | | | R122K, | |
| | | | T160E | |
| 224 | Lipase variant | N57P, K122R, A212P, | M110V, | NUC |
| | | S234K, D289G, G109H, | Y117F, | |
| | | | R122K, | |
| | | | T160E | |
| 225 | Lipase variant | N57P, K122R, A212P, | M110V, | PRT |
| | | S234K, D289G, G109H, | A111T, | |
| | | | Y117F, | |
| | | | R122K | |
| 226 | Lipase variant | N57P, K122R, A212P, | M110V, | NUC |
| | | S234K, D289G, G109H, | A111T, | |
| | | | Y117F, | |
| | | | R122K | |
| 227 | Lipase variant | N57P, K122R, A212P, | M110V, | PRT |
| | | S234K, D289G, G109H, | A111T, | |
| | | | K236T, | |
| | | | R238K | |
| 228 | Lipase variant | N57P, K122R, A212P, | M110V, | NUC |
| | | S234K, D289G, G109H, | A111T, | |
| | | | K236T, | |
| | | | R238K | |
| 229 | Lipase variant | N57P, K122R, A212P, | M110V, | PRT |
| | | S234K, D289G, G109H, | A111T | |
| 230 | Lipase variant | N57P, K122R, A212P, | M110V, | NUC |
| | | S234K, D289G, G109H, | A111T | |
| 231 | Lipase variant | N57P, K122R, A212P, | A111T, | PRT |
| | | S234K, D289G, G109H, | R122Q, | |
| | | | R238E | |
| 232 | Lipase variant | N57P, K122R, A212P, | A111T, | NUC |
| | | S234K, D289G, G109H, | R122Q, | |
| | | | R238E | |
| 233 | Lipase variant | N57P, K122R, A212P, | D54F, | PRT |
| | | S234K, D289G, G109H, | R122Q, | |
| | | | K236T, | |
| | | | R238E | |
| 234 | Lipase variant | N57P, K122R, A212P, | D54F, | NUC |
| | | S234K, D289G, G109H, | R122Q, | |
| | | | K236T, | |
| | | | R238E | |
| 235 | Lipase variant | N57P, K122R, A212P, | M110V, | PRT |
| | | S234K, D289G, G109H, | Y117F, | |
| | | | R122K, | |
| | | | K236T, | |
| | | | R238E | |
| 236 | Lipase variant | N57P, K122R, A212P, | M110V, | NUC |
| | | S234K, D289G, G109H, | Y117F, | |
| | | | R122K, | |
| | | | K236T, | |
| | | | R238E | |
| 237 | Lipase variant | N57P, K122R, A212P, | R122Q, | PRT |
| | | S234K, D289G, G109H, | T160E, | |
| | | | Y216K | |
| 238 | Lipase variant | N57P, K122R, A212P, | R122Q, | NUC |
| | | S234K, D289G, G109H, | T160E, | |
| | | | Y216K | |
| 239 | Lipase variant | N57P, K122R, A212P, | Y117F, | PRT |
| | | S234K, D289G, G109H, | R122K, | |
| | | | K236T | |
| 240 | Lipase variant | N57P, K122R, A212P, | Y117F, | NUC |
| | | S234K, D289G, G109H, | R122K, | |
| | | | K236T | |

### EXAMPLES

The following examples explain the present invention, but without restricting it.

### Starting Materials and Protocols

Analytical grade chemicals and ready-to-use kits were sourced and used from common suppliers such as Sigma Aldrich, Acros Organics, Fisher Scientific, Qiagen or Stratagene. Novozym® 435 is bought from Sigma Aldrich.

### Example 1

### Cloning of Lipase Variants:

Nucleic acid sequences of SEQ ID No. 1 or their variants were cloned into an expression vector based on pKA81. The genetic elements were incorporated into the vector by means known in the art. For expression of the different lipase variants, the vectors were introduced into electrocompetent Escherichia coli (E. coli) W3110 cells.

### Variant Library:

Nucleotides were substituted in the wild-type sequence or sequences derived therefrom to allow for amino acid substitution. This exchange can be achieved by various molecular biological methods. One method for replacing nucleic acids is site-directed mutagenesis, which can result in mutations at one or more sites in the amino acid sequence. Methods for site-directed mutagenesis are state of the art and are described in the literature (e.g. Directed Mutagenesis: A Practical Approach, 1991, Edited by M. J. McPHERSON, IRL PRESS) and can be purchased as ready-made kits (e.g. the QUIKCHANGE™ lightening mutagenesis kit from Qiagen or Stratagene). After inserting mutations into the gene sequence and culturing in a suitable E. coli cloning strain, the plasmids obtained were transformed into Escherichia coli W3110.

Transformed cells were tested in appropriate biotransformation reactions in order to determine product yield and selectivity. Appropriate biotransformation reactions are described below, see Example 2. Sequence verification was performed as known in the art.

Glycerol stocks of the E. coli cultures transformed with the respective expression plasmids were prepared by adding one volume of 40% glycerol solution to one volume of E. coli culture.

For isolation of single bacterial colonies, appropriate dilutions of E. coli cultures were plated onto LB-Agar plates containing suitable concentrations of kanamycin and incubated at 37° C. until single colonies were obtained.

### Example 2

### Cultivation:

To prepare precultures, sterilized 2 mL 96-well deep-well plates (Eppendorf, Hamburg, Germany) were used and mixed with 590 μL TB medium (50 μg/ml kanamycin) and 10 μL of the glycerol stock of the respective E. coli strain filled. Alternatively, 590 μL of TB medium (50 μg/ml kanamycin) were inoculated with cell material from a colony from an agar plate. The precultures were incubated at 37° C. and 250 rpm for 17 h.

Sterile 2 mL 96-well deep-wells with 510 μL TB medium (50 mg/L kanamycin) were used to produce main cultures. The expression cultures were inoculated with 30 μl of preculture and incubated at 37° C. and 250 rpm. After 4 h incubation, the enzyme expression was induced by adding 60 μL IPTG (10 mM IPTG diluted in expression medium, supplemented with 50 mg/L kanamycin). The expression culture plates were then incubated for 20 h at 28° C. and 250 rpm.

Cells were harvested by centrifugation for 15 min at 4° C. and 2500×g. The culture supernatant was discarded, and the cell pellet was suspended in 200 μL PBS. The cells were then lyophilized for 24 h and stored at 4° C. until use.

### Biotransformation, Chiral Resolution and Analytics:

The chiral resolution of methyl (3S)-3-hydroxy-2-methylene-butanoate was conducted in microtiter plates with the lyophilized supernatants. For the chiral resolution, 100 μl racemic methyl-3-hydroxy-2-methylene-butanoate, 105 μl MTBE (Methyl-tert-butylether) and 45 μl ultrapure water (with 416.6 g/L KHCO₃) were used in each well. Each plate was sealed and incubated for 6 h at 45° C. in a shaker at 300 rpm. The reaction was then stopped by adding 22.5 μl of 20% H₂SO₄ solution. For further extraction, 1 ml MTBE was added per well in the deep well plates. The plates were shaken at room temperature for 10 min and then centrifuged at 2500×g for 10 min. 10 μl of the organic phase were then transferred to 96-well PCR plates containing 100 μl MTBE and analyzed by HPLC.

### Analytical HPLC Method

The samples were analyzed using HPLC with the following settings:

Instrument: Agilent Technologies 1290 Infinity II; Column: Lux cellulose-2, 100×4.6 mm, 3 μm; Eluent A: premixed heptane (+0.05% formic acid); eluent B: ethanol; flow: isocratic (90% eluent A/10% eluent B), flow rate: 0.8 mL/min; temperature: 25° C.; sample injection volume: 1 μL; Detection: absorption at 210 nm.

Racemic methyl-3-hydroxy-2-methylene-butanoate and racemic 3-hydroxy-2-methylene-butanoic acid (prepared as for the biotransformation samples) were used as a reference substance and as a standard for quantification. Appropriate dilutions of the standards were used in order to be able to quantify the substrate used and the resulting product using standard lines. The samples were analyzed for substrate conversion and product formation. The evaluation and comparison of individual samples with one another was carried out by determining the substrate and product ee [%].

### Example 3: Enzymatic Hydrolysis of Racemic Methyl 3-hydroxy-2-methylene-butanoate to (3R)-3-hydroxy-2-methylene-butanoic Acid

The cultivation, biotransformation and HPLC analysis were carried out as described in example 2. During cultivation, glycerol cultures were used for inoculation. The activity and selectivity results of enzyme variants with individual point mutations can be found below in Tables 5 and 6. The activity and selectivity results of enzyme variants with combined mutations can be found in Tables 7 and 8. The activity and selectivity results of enzyme variants with additional combined mutations can be found in Tables 9 and 10.

A high selectivity and a high activity of the respective enzyme variant are decisive for an efficient conversion of racemic methyl 3-hydroxy-2-methylene-butanoate to (3R)-3-hydroxy-2-methylene-butanoic acid in order to generate an almost enantiomerically pure product in high yield.

The enzyme selectivity ee [%] 3-hydroxy-2-methylene-butanoate is defined by the difference of the mole fraction of (3R)-3-hydroxy-2-methylene-butanoic acid and (3S)-3-hydroxy-2-methylene-butanoic acid divided by the sum of the mole fraction of (3R)-3-hydroxy-2-methylene-butanoic acid and (3S)-3-hydroxy-2-methylene-butanoic acid.

$$\frac{\begin{matrix}( 3 R ) 3 hydroxy 2 methylenebutanoic acid - \\ ( 3 S ) 3 hydroxy 2 methylenebutanoic acid\end{matrix}}{\begin{matrix}( 3 R ) 3 hydroxy 2 methylenebutanoic acid + \\ ( 3 S ) 3 hydroxy 2 methylenebutanoic acid\end{matrix}} * 100$$

The enzyme activity ee [%] for the conversion of the mole fraction of the substrate methyl (3R) 3-hydroxy-2-methylene-butanoate is described by the difference between the mole fraction of methyl-(3S) 3-hydroxy-2-methylene-butanoate and methyl-(3R) 3-hydroxy-2-methylene-butanoate divided by the sum of methyl-(3S) 3-hydroxy-2-methylene-butanoate and methyl-(3R) 3-hydroxy-2-methylene-butanoate.

$$\frac{\begin{matrix}methyl ( 3 R ) 3 hydroxy 2 methylenebutanoate - \\ methyl ( 3 S ) 3 hydroxy 2 methylenebutanoate\end{matrix}}{\begin{matrix}methyl ( 3 R ) 3 hydroxy 2 methylenebutanoate + \\ methyl ( 3 S ) 3 hydroxy 2 methylenebutanoate\end{matrix}} * 100$$

The lipase of SEQ ID No. 1 shows a selectivity of 100% and an activity of 100%.

TABLE 5
Lipase variants showing a relative improvement in enzyme activity
compared to the lipase of SEQ ID No. 1. The relative improvement
in enzyme activity is defined as the quotient of the enantiomeric
excess ee[%] of the respective variant and the enantiomeric excess
ee[%] of the reference lipase (SEQ ID No.1) in percent. The substrate
ee[%] of the lipase of SEQ ID No. 1 is 9.8.

| | Relative improvement |
|---|---|
| Reference | in enzyme activity |
| lipase/ | compared to reference |
| variant | lipase of Seq ID No: 1 |
| G109H | 263.1 |
| L44M | 179.8 |
| L44W | 178.4 |
| K122Q | 172.6 |
| L44Y | 151.6 |
| A184G | 150.8 |
| A212S | 127.5 |
| Reference | 100.0 |
| Lipase (SEQ | |
| ID No. 1) | |

TABLE 6
Lipase variants showing a relative improvement in enzyme
selectivity compared to the reference lipase of SEQ ID No. 1.
The relative improvement in enzyme selectivity is defined
as the quotient of ee[%] 3-hydroxy-2-methylene-butanoic
acid of the respective variant and the ee[%] of
3-hydroxy-2-methylene-butanoic acid of the reference lipase
of SEQ ID No. 1 in percent. The product ee[%] of the
reference lipase is 75.8.

| | Relative improvement in |
|---|---|
| | enzyme selectivity compared |
| Variant | to Enzyme of Seq ID No: 1 |
| L44W | 128.5 |
| Y117F | 127.3 |
| L44Y | 126.2 |
| Y216K | 117.5 |
| G109H | 117.1 |
| Y117S | 115.8 |
| M110T | 115.4 |
| T160E | 114.8 |
| L321F | 114.7 |
| M110V | 114.6 |
| A181Q | 114.3 |
| T160C | 113.9 |
| Y240F | 113.5 |
| T160D | 112.9 |
| T160P | 112.9 |
| T160I | 112.7 |
| T160Q | 112.4 |
| T160K | 112.0 |
| T160M | 111.7 |
| D54Q | 111.6 |
| D289S | 111.1 |
| D54M | 111.1 |
| T53S | 110.8 |
| R238A | 110.5 |
| R238K | 110.5 |
| N320E | 110.5 |
| H153N | 110.1 |
| D54F | 110.0 |
| K122A | 109.8 |
| R238D | 109.7 |
| D54G | 109.6 |
| N317T | 109.4 |
| T160S | 108.9 |
| R238E | 108.8 |
| K122Q | 108.8 |
| T53P | 108.6 |
| R238Q | 108.4 |
| Y216A | 107.9 |
| S234K | 107.5 |
| T160F | 107.3 |
| H153Y | 107.3 |
| L44M | 107.2 |
| D54E | 107.1 |
| G291E | 107.0 |
| T53I | 107.0 |
| G291A | 106.8 |
| D54L | 106.6 |
| T160A | 106.5 |
| D54T | 106.5 |
| A212S | 106.4 |
| A55R | 106.0 |
| H153D | 105.9 |
| T53E | 105.9 |
| G109F | 105.8 |
| A55M | 105.7 |
| T53A | 105.5 |
| T160N | 105.5 |
| N320G | 105.2 |
| F51N | 105.1 |
| F51M | 104.9 |
| D179C | 104.7 |
| D289G | 104.7 |
| A55D | 104.6 |
| A111S | 104.6 |
| A55Y | 104.2 |
| V52L | 104.1 |
| K236T | 104.1 |
| H153E | 104.0 |
| S234T | 103.9 |
| K122Y | 103.9 |
| K122R | 103.4 |
| Y121V | 103.2 |
| A184T | 103.0 |
| A55S | 102.9 |
| H153C | 102.8 |
| D54P | 102.6 |
| K122V | 102.3 |
| A184G | 102.2 |
| Y211E | 101.6 |
| S234G | 101.2 |
| A55I | 101.0 |
| Reference | 100.0 |
| lipase SEQ ID | |
| NO. 1 | |

TABLE 7
Lipase variants showing a relative improvement in enzyme activity
compared to the reference lipase of SEQ ID No. 1. The relative
improvement in enzyme activity is defined as the quotient of the
enantiomeric excess ee[%] of the respective variant and the
enantiomeric excess ee[%] of the reference lipase
(SEQ ID No. 1) in percent.

| | Relative improvement in |
|---|---|
| | enzyme activity compared |
| | to reference lipase of |
| Variant | SEQ ID NO. 1 |
| N57P, G109H, K122R, A212P, | 264.7 |
| S234K, D289G (SEQ ID NO. 171) | |
| Reference lipase SEQ ID NO. 1 | 100.0 |

TABLE 8
Lipase variants showing a relative improvement in enzyme
selectivity compared to the reference lipase of SEQ ID No. 1.
The relative improvement in enzyme selectivity is defined as
the quotient of ee[%] 3-hydroxy-2-methylene-butanoic acid
of the respective variant and the ee[%] of 3-hydroxy-2-methylene-
butanoic acid of the reference lipase of SEQ ID No. 1 in percent.

| | Relative improvement |
|---|---|
| | in enzyme selectivity |
| | compared to reference |
| Variant/Reference lipase | lipase of SEQ ID No. 1 |
| N57P, G109H, K122R, A212P, S234K, D289G | 102.5 |
| (SEQ ID NO. 171) | |
| N57P, K122R, A212P, S234K, D289G | 110.1 |
| (SEQ ID NO. 169) | |
| Reference lipase SEQ ID NO. 1 | 100.0 |

### Example 4

Variants based on the backbone mutations N57P, K122R, A212P, S234K, D289G and G109H have been supplemented with further mutations in the sequence as described in the examples above and tested for enzyme activity and selectivity as described in Example 3.

TABLE 9
Lipase variants showing a relative improvement in enzyme activity compared to the
reference lipase of SEQ ID No. 171. The relative improvement in enzyme activity is
defined as the quotient of the enantiomeric excess ee[%] of the respective variant and
the enantiomeric excess ee[%] of the reference lipase (SEQ ID No. 171) in percent.
All lipase variants in the table carry in addition the mutations N57P, K122R, A212P,
S234K, D289G, G109H.

| | Relative improvement in enzyme |
|---|---|
| | activity compared to variant |
| | N57P, G109H, K122R, A212P, |
| Variant/Reference lipase | S234K, D289G (SEQ ID No. 171) |
| A55R, N57P, G109H, K122R, A212P, S234K, | 134.2 |
| K236T, R238K, D289G | |
| LA4Y, N57P, G109H, K122R, T160E, A212P, | 131.7 |
| S234K, K236T, D289G | |
| L44W, D54F, N57P, G109H, K122R, T160E, | 128.5 |
| A212P, S234K, K236T, R238E, D289G | |
| LA4Y, N57P, G109H, K122R, T160E, A212P, | 125.9 |
| S234K, K236T, Y240F, D289G | |
| N57P, G109H, M110T, K122R, A212P, S234K, | 125.2 |
| K236T, Y240F, D289G | |
| N57P, G109H, K122R, T160E, A212P, S234K, | 124.6 |
| Y240F, D289G | |
| LA4Y, N57P, G109H, K122R, A212P, S234K, | 124.4 |
| K236T, R238K, D289G | |
| LA4Y, A55R, N57P, G109H, K122R, A212P, | 123.4 |
| S234K, K236T, D289G | |
| LA4Y, N57P, G109H, K122R, A212P, S234K, | 123.4 |
| R238E, D289G | |
| Reference lipase of SEQ ID No.171 | 100.0 |

TABLE 10
Lipase variants showing a relative improvement in enzyme selectivity compared
to the reference lipase of SEQ ID No. 171. The relative improvement in enzyme
selectivity is defined as the quotient of ee[%] 3-hydroxy-2-methylene-butanoic
acid of the respective variant and the ee[%] of 3-hydroxy-2-methylene-butanoic
acid of the reference lipase of SEQ ID No. 171 in percent. All lipase variants in the
table carry in addition the mutations N57P, K122R, A212P, S234K, D289G, G109H.

| | Relative improvement |
|---|---|
| | in enzyme selectivity |
| | compared to K122R, |
| | N57P, G109H, |
| | variant D289G |
| | A212P, S234K, |
| Variant/Reference lipase | (SEQ ID No. 171) |
| N57P, G109H, M110T, A111T, K122R, A212P, S234K, | 101.2 |
| D289G | |
| N57P, G109H, M110T, K122R, A212P, S234K, D289G | 101.1 |
| D54F, N57P, G109H, M110V, K122R, T160E, A212P, | 101.1 |
| S234K, D289G | |
| LA4W, N57P, G109H, M110T, A111T, K122Q, A212P, | 101.1 |
| S234K, D289G | |
| N57P, G109H, M110V, K122R, A212P, S234K, K236T, | 101.0 |
| R238E, D289G | |
| N57P, G109H, A111T, K122Q, A212P, S234K, R238E, | 101.0 |
| D289G | |
| N57P, G109H, M110V, A111T, K122R, A212P, S234K, | 101.0 |
| K236T, R238K, D289G | |
| N57P, G109H, M110V, A111T, Y117F, A212P, S234K, | 101.0 |
| D289G | |
| N57P, G109H, K122Q, T160E, A212P, Y216K, S234K, | 101.0 |
| D289G | |
| N57P, G109H, K122R, T160E, A212P, S234K, D289G | 101.0 |
| N57P, G109H, M110V, Y117F, A212P, S234K, K236T, | 101.0 |
| R238E, D289G | |
| N57P, G109H, M110V, A111T, K122R, A212P, S234K, | 101.0 |
| D289G | |
| N57P, G109H, M110V, Y117F, T160E, A212P, S234K, | 101.0 |
| D289G | |
| D54F, N57P, G109H, Y117F, T160E, A212P, S234K, | 101.0 |
| D289G | |
| N57P, G109H, Y117F, A212P, S234K, K236T, R238K, | 100.9 |
| D289G | |
| N57P, G109H, K122R, A212P, S234K, R238E, D289G | 100.9 |
| N57P, G109H, A212P, S234K, R238E, D289G | 100.9 |
| N57P, G109H, M110V, K122R, A212P, S234K, D289G | 100.9 |
| N57P, G109H, T160E, A212P, S234K, D289G | 100.9 |
| N57P, G109H, Y117F, K122Q, A212P, S234K, K236T, | 100.9 |
| R238E, D289G | |
| N57P, G109H, Y117F, A212P, S234K, K236T, D289G | 100.9 |
| N57P, G109H, K122R, T160E, A212P, S234K, R238E, | 100.8 |
| D289G | |
| N57P, G109H, K122R, A212P, S234K, Y240F, D289G | 100.7 |
| D54F, N57P, G109H, K122Q, A212P, S234K, K236T, | 100.7 |
| R238E, D289G | |
| Reference lipase of SEQ ID No.171 | 100.0 |

### Example 5: Enzymatic Hydrolysis of Different Esters

Next to a methyl ester of 3-hydroxy-2-methylene-butanoate (a), also the enzymatic hydrolysis of the respective tert-butyl ester (b), iso-butyl-ester (c), and iso-propyl ester (d) were tested under suitable conditions (1 mL scale, 5 g/L ester compound, 5 g/L lyophilizate of enzyme of SEQ ID No. 1, 100 mM KPi buffer pH 8):

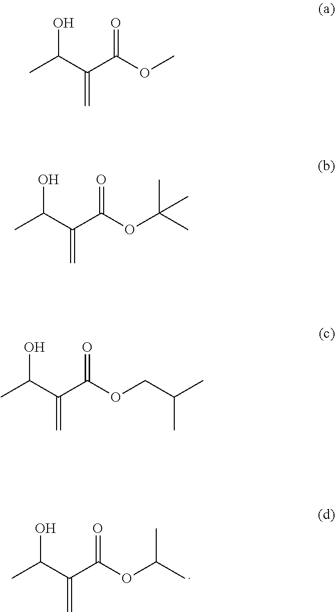

All esters (a) to (d) allow for an efficient enantiomeric resolution, wherein the (R)-Ester is preferably hydrolysed:

| | time | | ee | loss of (S)- | loss of (R)- |
|---|---|---|---|---|---|
| ester | [h] | ° C. | [%] | ester [%]* | ester [%]* |
| methyl | 1 | 30 | 98.9 | <16 | >99 |
| Tert-butyl | 20 | 30 | 92.5 | <5 | >95 |
| Iso-butyl | 1 | 30 | 56.8 | <50 | <85 |
| Iso-propyl | 0.25 | 45 | 100 | <50 | >99 |

*compared to timepoint-zero control

### Example 6: Alternative Solvents

Several different solvents have been tested for the chiral resolution of methyl 3-hydroxy-2-methylene-butanoate. Reaction conditions were: 100 mL total volume, 300 g/L racemic methyl 3-hydroxy-2-BCSmethylene-butanoate, 20 g/L spray dried lipase of SEQ ID No. 1, 70% solvent, 0.54 eq KHCO₃, pH 8.5 (titration with 40% w/v K₂CO₃), 45° C., 6 h. In particular MTBE, CPME, MIBK and toluene allowed for a high enantiomeric excess of the hydrolysed product (3R)-3-hydroxy-2-methylene-butanoic acid (ee).

| | ee | c |
|---|---|---|
| Solvent | (%) | (%) |
| MTBE (Methyl-tert- | 97 | 53 |
| butylether) | | |
| MeTHF (2- | 24 | 24 |
| Methyltetrahydrofuran) | | |
| CPME (Cyclopentyl | 88 | 58 |
| methyl ether) | | |
| MIBK (Methyl isobutyl | 89 | 58 |
| ketone) | | |
| Cyclohexane | 39 | 37 |
| Toluene | 89 | 65 |

### Example 7: Further Examples on Enzymatic Hydrolysis of Racemic Methyl 3-hydroxy-2-methylene-butanoate to (3R)-3-hydroxy-2-methylene-butanoic Acid

Next to the lipase of SEQ ID No.1, three further related variants have been tested, namely variants with mutations Q295V and K298A (SEQ ID No. 3); as well as with mutations V331 and 1254S (SEQ ID No. 5); as well as with mutations T188S, I254S, P302L and Q304E (SEQ ID No. 241).

The cultivation, biotransformation and HPLC analysis were carried out as described in example 2. As described in example 3, the enzyme selectivity ee [%] 3-hydroxy-2-methylene-butanoate is defined by the difference of (3R)-3-hydroxy-2-methylene-butanoic acid and (3S)-3-hydroxy-2-methylene-butanoic acid divided by the sum of (3R)-3-hydroxy-2-methylene-butanoic acid and (3S)-3-hydroxy-2-methylene-butanoic acid. The enzyme activity ee [%] for the conversion of the substrate methyl (3R) 3-hydroxy-2-methylene-butanoate is described by the difference between methyl-(3S) 3-hydroxy-2-methylene-butanoate and methyl-(3R) 3-hydroxy-2-methylene-butanoate divided by the sum of methyl-(3S) 3-hydroxy-2-methylene-butanoate and methyl-(3R) 3-hydroxy-2-methylene-butanoate. The reference lipase of SEQ ID No. 1 shows a selectivity of 100% and an activity of 100%

TABLE 11
Comparative lipase variants showing a similar enzyme activity
profile compared to the reference lipase of SEQ ID No. 1. The
relative difference in enzyme activity is defined as the quotient
of the enantiomeric excess ee[%] of the respective variant and the
enantiomeric excess ee[%] of the reference lipase (SEQ ID No. 1)
in percent.

| | Relative difference in |
|---|---|
| | enzyme activity compared |
| Reference | to reference lipase |
| lipase/variant | of Seq ID No: 1 |
| Q295V and | 78.2 |
| K298A | |
| V33I and | 53.8 |
| I254S | |
| T188S, | |
| I254S, | |
| P302L and | |
| Q304E | 43.4 |
| Reference | 100.0 |
| Lipase (SEQ | |
| ID No.1) | |

TABLE 12
Comparative lipase variants showing a similar enzyme selectivity
compared to the reference lipase of SEQ ID No. 1. The relative
difference in enzyme selectivity is defined as the quotient of
ee[%] 3-hydroxy-2-methylene-butanoic acid of the respective
variant and the ee[%] of 3-hydroxy-2-methylene-butanoic acid
of the reference lipase of SEQ ID No. 1 in percent.

| | Relative difference |
|---|---|
| | in enzyme selectivity |
| | compared to reference |
| Variant | lipase of Seq ID No: 1 |
| Q295V and | 100.1 |
| K298A | |
| V33I and | 99.1 |
| I254S | |
| T188S, I254S, | 102.5 |
| P302L and | |
| Q304E | |
| Reference | 100.0 |
| Lipase (SEQ | |
| ID No.1) | |

As shown in the Tables 11 und 12, the comparative lipases worsen the activity of the lipase, and do not improve the selectivity of the discussed substrate.

### Example 8: Enzymatic Hydrolysis of Racemic Methyl 3-hydroxy-2-methylene-butanoate to (3R)-3-hydroxy-2-methylene-butanoic Acid

8.0 g spray dried lipase SEQ ID No. 1 were suspended in 50 g water and diluted with 100 mL MTBE. After warming to 45° C. a mixture of 120 g racemic methyl 3-hydroxy-2-methylene-butanoate (99% purity) in 100 g MTBE were dosed into the suspension over 3 h. After full dosage, the mixture was further stirred at 45° C. over night. During dosage and additional stirring time, the pH was kept at 7.5 by the parallel addition of 40% aqueous potassium carbonate solution. Afterwards, the biomass was separated by centrifugation and the phases were separated. After extraction of the aqueous phase with 2× 125 g MTBE each, the combined organic extracts were concentrated under reduced pressure at 45° C. yielding methyl (3S)-3-hydroxy-2-methylene-butanoate with 91% purity, 96% ee and and corrected yield of 40%.

### Example 9: Enzymatic Hydrolysis of Racemic Methyl 3-hydroxy-2-methylene-butanoate to (3R)-3-hydroxy-2-methylene-butanoic acid

120 g water were warmed to 45° C. and 2.0 g spray dried lipase SEQ ID No. 1 were added, followed by 280 mL MTBE. At 45° C., 120 g racemic methyl 3-hydroxy-2-methylene-butanoate (99% purity) were dosed into the suspension over 2 h. After full dosage, the mixture was further stirred at 45° C. for 8.5h. During dosage and additional stirring time, the pH was kept at 7.5 by the parallel addition of 40% aqueous potassium carbonate solution. Afterwards, the lower aqueous phase was separated and back-extracted once with 100 ml MTBE. The extract was combined with the upper organic phase and the mixture was distilled at 33-55° C. and reduced pressure down to 10 mbar. After addition of 50 g high boiling Marlotherm, the residue was further distilled at 3-10 mbar up to 150° C. yielding 58.2 g (92% purity, 96% ee, 45% yield) methyl (3S)-3-hydroxy-2-methylene-butanoate with 91% purity, 96% ee and and corrected yield of 40%.

### Example 10: Enantioselective Acylation of Racemic methyl-3-hydroxy-2-methylene-butanoate

A suspension of 1400 g racemic methyl 3-hydroxy-2-methylene-butanoate [10.62 mol, 98.7% purity] and 105 g Novozyme 435 is heated to 25° C. internal temperature. 1373 g vinyl dodecanoate (5.95 mol, 98%) is added in 3 h using a dosing pump under 50 mbar vacuum. Subsequently, the reaction mixture heated to 35° C. internal temperature and 50 mbar for additional 8h. Acetaldehyde is distilled out under vacuum. Afterwards the suspension is allowed to proceed for additional 8 h at 35° C. and 50 mbar. The reaction mixture is heated to 115° C. jacket temperature under vacuum to distill methyl (3S)-3-hydroxy-2-methylene-butanoate out of the suspension. In that respect, the vacuum is gradually reduced to 3 mbar and the jacket temperature is increased to 135° C. The product is analyzed by using chiral HPLC standard method. A chemical purity of >99% is and an enantiomeric excess of >98% ee is achieved. The isolated yield of methyl (3S)-3-hydroxy-2-methylene-butanoate is 41%.

1. A protein variant having lipase activity wherein the protein variant is encoded by an amino acid sequence having at least 80% identity with an amino acid sequence shown under SEQ ID No. 1,
wherein the amino acid sequence of the protein variant differs from the amino acid sequence of SEQ ID NO. 1 in at least one of the following positions:
i. the amino acid at position 44 is M, W or Y;
ii. the amino acid at position 51 is N, or M;
iii. the amino acid at position 52 is L;
iv. the amino acid at position 53 is S, P, I, E, or A;
v. the amino acid at position 54 is Q, M, F, G, E, L, T or P;
vi. the amino acid at position 55 is R, M, D, Y, S, I;
vii. the amino acid at position 109 is H or F;
viii. the amino acid at position 110 is T or V;
ix. the amino acid at position 111 is S;
x. the amino acid at position 117 is F or S;
xi. the amino acid at position 121 is V;
xii. the amino acid at position 122 is Q, A, Y, R, or V;
xiii. the amino acid at position 153 is Y, D, E, or C;
xiv. the amino acid at position 160 is E, C, D, P, I, Q, K, M, S, F, A, or N;
xv. the amino acid at position 179 is C;
xvi. the amino acid at position 181 is Q;
xvii. the amino acid at position 184 is G or T;
xviii. the amino acid at position 211 is E;
xix. the amino acid at position 212 is S;
xx. the amino acid at position 216 is K or A;
xxi. the amino acid at position 234 is K, T or G;
xxii. the amino acid at position 235 is V or M;
xxiii. the amino acid at position 236 is T;
xxiv. the amino acid at position 238 is A, K, D, E, or Q;
xxv. the amino acid at position 240 is F;
xxvi. the amino acid at position 289 is S or G;
xxvii. the amino acid at position 291 is E or W;
xxviii. the amino acid at position 317 is T;
xxix. the amino acid at position 320 is E or G;
xxx. the amino acid at position 321 is F.

2. The protein variant according to claim 1, wherein the amino acid sequence of the protein variant differs from the amino acid sequence of SEQ ID NO. 1 by at least one of the following mutations:
i. the amino acid at position 44 is W or Y;
ii. the amino acid at position 54 is F;
iii. the amino acid at position 55 is R;
iv. the amino acid at position 109 is H;
v. the amino acid at position 110 is T or V;
vi. the amino acid at position 117 is F;
vii. the amino acid at position 122 is Q or R;
viii. the amino acid at position 160 is E;
ix. the amino acid at position 216 is K;
x. the amino acid at position 236 is T;
xi. the amino acid at position 238 is K or E;
xii. the amino acid at position 240 is F.

3. The protein variant according to claim 1, wherein the amino acid sequence of the protein variant differs from the amino acid sequence of SEQ ID NO. 1 by at least the following mutation: the amino acid at position 109 is H.

4. The protein variant according to claim 1, wherein the protein variant comprises at least two of the recited amino acid substitutions.

5. The protein variant according to claim 1, wherein the amino acid sequence of the protein variant differs from the amino acid sequence of SEQ ID NO. 1 by at least two of the following modifications selected from:
i. the amino acid at position 57 is P;
ii. the amino acid at position 109 is H;
iii. the amino acid at position 122 is R;
iv. the amino acid at position 212 is P;
v. the amino acid at position 234 is K;
vi. the amino acid at position 289 is G.

6. The protein variant according to claim 1, wherein the amino acid sequence of the protein variant differs from the amino acid sequence of SEQ ID NO. 1 by the following modifications:
i. the amino acid at position 57 is P; the amino acid at position 109 is H; the amino acid at position 122 is R or K or Q, preferably R; the amino acid at position 212 is P; the amino acid at position 234 is K; the amino acid at position 289 is G;
ii. and additionally at least one of the following modifications: the amino acid at position 44 is W or Y; the amino acid at position 54 is F, the amino acid at position 55 is R, the amino acid at position 110 is V or T; the amino acid at position 111 is T, the amino acid at position 117 is F, the amino acid at position 160 is E, the amino acid at position 216 is K, the amino acid at position 236 is T, the amino acid at position 238 is K or E, the amino acid at position 240 is F.

7. The protein variant according to claim 1, wherein the protein variant carries an amino acid sequence of one of SEQ ID No. 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, and 239.

8. A nucleic acid molecule encoding a protein having lipase activity according to claim 1.

9. The nucleic acid molecule according to claim 8 encoding a protein having lipase activity selected from the group consisting of
a) nucleic acid molecules comprising nucleic acid sequences shown under of SEQ ID No. 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, and 240;
b) nucleic acid molecules having at least 60% identity with the nucleic acid sequences shown under a).

10. A recombinant nucleic acid molecule comprising a nucleic acid molecule according to claim 8.

11. The recombinant nucleic acid molecule according to claim 10, wherein the recombinant nucleic acid molecule is a vector or a plasmid.

12. A host cell comprising a protein according to claim 1.

13. (canceled)

14. A method for hydrolysing a substrate of formula (II) in an enantiomerically selective manner to a compound of formula (III), the method comprising contacting the substrate with a protein variant according to claim 1, 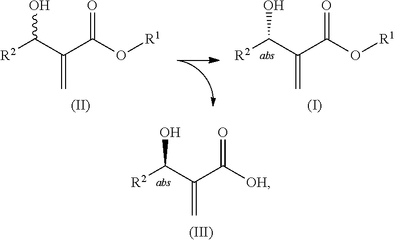
wherein R¹ and R² are independently selected from a substituted or unsubstituted (n)-alkyl, iso-alkyl, aryl, alkyl-substituted or aryl-substituted aryl.

15. The method according to claim 14 for hydrolysing a substrate of formula (II-1) in an enantiomerically selective manner to a compound of formula (III-1), 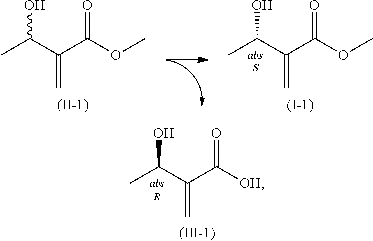
wherein the method further comprises separating a compound of formula (I-1).

16. The method according to claim 15, wherein the method is carried out in a biphasic system of water and an organic solvent.

17. The protein variant according to claim 4, wherein the protein variant comprises at least three of the recited amino acid substitutions.

18. The protein variant according to claim 5, wherein the amino acid sequence of the protein variant differs from the amino acid sequence of SEQ ID NO. 1 by four of the following modifications selected from:
vii. the amino acid at position 57 is P;
viii. the amino acid at position 109 is H;
ix. the amino acid at position 122 is R;
x. the amino acid at position 212 is P;
xi. the amino acid at position 234 is K;
xii. the amino acid at position 289 is G.

19. The protein variant according to claim 18, wherein the amino acid sequence of the protein variant differs from the amino acid sequence of SEQ ID NO. 1 by all of the following modifications selected from:
xiii. the amino acid at position 57 is P;
xiv. the amino acid at position 109 is H;
xv. the amino acid at position 122 is R;
xvi. the amino acid at position 212 is P;
xvii. the amino acid at position 234 is K;
xviii. the amino acid at position 289 is G.

20. The nucleic acid molecule according to claim 9 encoding the protein having lipase activity selected from the group consisting of
a) nucleic acid molecules comprising nucleic acid sequences shown under of SEQ ID No. 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, and 240;
b) nucleic acid molecules having at least 90% identity with the nucleic acid sequences shown under a).

21. The method according to claim 16, wherein the organic solvent is selected from methyl-tert-butylether, toluene, 2-methyltetrahydrofuran, methyl isobutyl ketone, cyclohexane, cyclopentyl methyl ether, chlorobenzene, tert-amylmethylether, ethyl acetate, and isopropyl acetate.


SEQUENCE LISTING (242 sequences)

SEQ ID NO: 1 - protein, 339 aa, unidentified
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 2 - DNA, 1017 bp, unidentified
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 3 - protein, 339 aa, Moraxella osloensis
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEVLQAAGAPSQLVHFADMPHAFLNMENLCPEQCKALYQKIGEFCQA

SEQ ID NO: 4 - DNA, 1017 bp, unidentified
  source 1..1017: mol_type = unassigned DNA
atgcccgagaacctgaagcagcccatgcccgccgccatgcaggcctacctgacccagctgcaccaggtgatcatcccctacctgctggcccagggcgtgaagcccaacgccatcaacgccagggaggccctggccaacctgaccaccgccttcgtgaccgacgcccccaacatggccaagatcatcgacaccgtgctggtgacccccagcaacttcaggggctacaacgtgcccctgaggctgttcgtgcccagcaactgccccctgcccaacgagcaggccaccgccgacgtgcccgtgatggtgtacttccacggcggcggcggcatggccggcagcgtgaccgtgtacgacaagctgtacaagaagctggccgaggccaccggctgcgtggtggtggcccccgagtacaggctggcccccgagaacaggtaccccgccgccatcgacgacgcccacgccatcctgacctacctgaccgccaccctgcagcaggtgggctacgcctgcaacggcaagctgatcatcgccggcgacagcgccggcggcgccatcaccgccaccctggtgcaggactggctggccaacagggtgagcaccgacctggccatcacccaccagatcctgatctacgccagcctggactacaccatgagccagcccagcatcgaggagaacggcaccggctacctgctggagagcagcaagatcaggtggtacttcgacaactacttcagcgcctacgacgacagggagctgatcagccccttctggaccgacatggccaccctgctgcaccacagcccctaccaggcccccaagaccctgcacctgaccgccggctactgccccctgagggacgaggacatcggctaccacgaggtgctgcaggccgccggcgcccccagccagctggtgcacttcgccgacatgccccacgccttcctgaacatggagaacctgtgccccgagcagtgcaaggccctgtaccagaagatcggcgagttctgccaggcc

SEQ ID NO: 5 - protein, 339 aa, Moraxella osloensis
MPENLKQPMPAAMQAYLTQLHQVIIPYLLAQGIKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELSSPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 6 - DNA, 1017 bp, unidentified
  source 1..1017: mol_type = other DNA
atgcccgagaacctgaagcagcccatgcccgccgccatgcaggcctacctgacccagctgcaccaggtgatcatcccctacctgctggcccagggcatcaagcccaacgccatcaacgccagggaggccctggccaacctgaccaccgccttcgtgaccgacgcccccaacatggccaagatcatcgacaccgtgctggtgacccccagcaacttcaggggctacaacgtgcccctgaggctgttcgtgcccagcaactgccccctgcccaacgagcaggccaccgccgacgtgcccgtgatggtgtacttccacggcggcggcggcatggccggcagcgtgaccgtgtacgacaagctgtacaagaagctggccgaggccaccggctgcgtggtggtggcccccgagtacaggctggcccccgagaacaggtaccccgccgccatcgacgacgcccacgccatcctgacctacctgaccgccaccctgcagcaggtgggctacgcctgcaacggcaagctgatcatcgccggcgacagcgccggcggcgccatcaccgccaccctggtgcaggactggctggccaacagggtgagcaccgacctggccatcacccaccagatcctgatctacgccagcctggactacaccatgagccagcccagcatcgaggagaacggcaccggctacctgctggagagcagcaagatcaggtggtacttcgacaactacttcagcgcctacgacgacagggagctgagcagccccttctggaccgacatggccaccctgctgcaccacagcccctaccaggcccccaagaccctgcacctgaccgccggctactgccccctgagggacgaggacatcggctaccacgagcagctgcagaaggccggcgcccccagccagctggtgcacttcgccgacatgccccacgccttcctgaacatggagaacctgtgccccgagcagtgcaaggccctgtaccagaagatcggcgagttctgccaggcc

SEQ ID NO: 7 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGHMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 8 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 9 - protein, 339 aa, synthetic construct
MPENLKQPMPAAMQAYLTQLHQVIIPYLLAQGVKPNAINAREAMANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 10 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccatggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 11 - protein, 339 aa, synthetic construct
MPENLKQPMPAAMQAYLTQLHQVIIPYLLAQGVKPNAINAREAWANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 12 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagcctgggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 13 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYQKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 14 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtaccagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 15 - protein, 339 aa, synthetic construct
MPENLKQPMPAAMQAYLTQLHQVIIPYLLAQGVKPNAINAREAYANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 16 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagcctatgcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 17 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGGITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 18 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgggattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 19 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYSSLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 20 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctactcgtctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 21 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVFDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 22 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctttgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 23 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDKTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 24 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggacaaaacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 25 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVSDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 26 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctcggacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 27 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGTAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 28 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtacggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 29 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLEATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 30 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 31 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENFCPEQCKALYQKIGEFCQA

SEQ ID NO: 32 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacttttgtccggaacagtgcaaagcgctgtatcagaaaattggcgagttttgccaagcg

SEQ ID NO: 33 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGVAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 34 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtgtggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 35 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSQGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 36 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctcagggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 37 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLCATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 38 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgtgcgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 39 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWFFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 40 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtttttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 41 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLDATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 42 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttggatgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 43 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLPATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 44 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgccggctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 45 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLIATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 46 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgattgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 47 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLQATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 48 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgcaggctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 49 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLKATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 50 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 51 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLMATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 52 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgatggctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 53 - protein, 339 aa, synthetic construct
MPENLKQPMPAAMQAYLTQLHQVIIPYLLAQGVKPNAINAREALANLTTAFVTQAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 54 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactcaggccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 55 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDESIGYHEQLQKAGAPSQLVHFADMPHAFLNMENLCPEQCKALYQKIGEFCQA

SEQ ID NO: 56 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgagagcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 57 - protein, 339 aa, synthetic construct
MPENLKQPMPAAMQAYLTQLHQVIIPYLLAQGVKPNAINAREALANLTTAFVTMAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 58 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactatggccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 59 - protein, 339 aa, synthetic construct
MPENLKQPMPAAMQAYLTQLHQVIIPYLLAQGVKPNAINAREALANLTTAFVSDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 60 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgagcgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 61 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIAWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 62 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatcgcgtggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 63 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIKWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 64 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatcaaatggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 65 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMEELCPEQCKALYQKIGEFCQA

SEQ ID NO: 66 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggaggaactttgtccggaacagtgcaaagcgctgtatcagaaaattggcgagttttgccaagcg

SEQ ID NO: 67 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDANAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 68 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcaaacgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 69 - protein, 339 aa, synthetic construct
MPENLKQPMPAAMQAYLTQLHQVIIPYLLAQGVKPNAINAREALANLTTAFVTFAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 70 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgacttttgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 71 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYAKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 72 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacgcgaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 73 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIDWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 74 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatcgattggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 75 - protein, 339 aa, synthetic construct
MPENLKQPMPAAMQAYLTQLHQVIIPYLLAQGVKPNAINAREALANLTTAFVTGAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 76 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactggtgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 77 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLTMENLCPEQCKALYQKIGEFCQA

SEQ ID NO: 78 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaccatggagaacctttgtccggaacagtgcaaagcgctgtatcagaaaattggcgagttttgccaagcg

SEQ ID NO: 79 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLSATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 80 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgagcgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 81 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIEWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 82 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatcgaatggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 83 - protein, 339 aa, synthetic construct
MPENLKQPMPAAMQAYLTQLHQVIIPYLLAQGVKPNAINAREALANLTTAFVPDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 84 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgccggatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 85 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIQWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 86 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccagtggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 87 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDATMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 88 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggacgcgacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 89 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLEKSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 90 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaaaatcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 91 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLFATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 92 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgtttgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 93 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAYAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 94 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcatatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 95 - protein, 339 aa, synthetic construct
MPENLKQPMPAAMQAYLTQLHQVIIPYLLAQGVKPNAINAREALANLTTAFVTEAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 96 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgaagccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 97 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIEYHEQLQKAGAPSQLVHFADMPHAFLNMENLCPEQCKALYQKIGEFCQA

SEQ ID NO: 98 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgaataccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 99 - protein, 339 aa, synthetic construct
MPENLKQPMPAAMQAYLTQLHQVIIPYLLAQGVKPNAINAREALANLTTAFVIDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 100 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgattgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 101 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIAYHEQLQKAGAPSQLVHFADMPHAFLNMENLCPEQCKALYQKIGEFCQA

SEQ ID NO: 102 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgcgtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 103 - protein, 339 aa, synthetic construct
MPENLKQPMPAAMQAYLTQLHQVIIPYLLAQGVKPNAINAREALANLTTAFVTLAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 104 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactctggccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 105 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLAATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 106 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttggctgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 107 - protein, 339 aa, synthetic construct
MPENLKQPMPAAMQAYLTQLHQVIIPYLLAQGVKPNAINAREALANLTTAFVTTAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 108 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactaccgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 109 - protein, 339 aa, synthetic construct
MPENLKQPMPAAMQAYLTQLHQVIIPYLLAQGVKPNAINAREALANLTTAFVTDRPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 110 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatcggccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 111 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDADAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 112 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcagatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 113 - protein, 339 aa, synthetic construct
MPENLKQPMPAAMQAYLTQLHQVIIPYLLAQGVKPNAINAREALANLTTAFVEDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 114 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtggaggatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 115 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGFMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 116 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggttttatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 117 - protein, 339 aa, synthetic construct
MPENLKQPMPAAMQAYLTQLHQVIIPYLLAQGVKPNAINAREALANLTTAFVTDMPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 118 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatatgccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 119 - protein, 339 aa, synthetic construct
MPENLKQPMPAAMQAYLTQLHQVIIPYLLAQGVKPNAINAREALANLTTAFVADAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 120 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtggcggatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 121 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLNATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 122 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaatgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 123 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMEGLCPEQCKALYQKIGEFCQA

SEQ ID NO: 124 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggaggggctttgtccggaacagtgcaaagcgctgtatcagaaaattggcgagttttgccaagcg

SEQ ID NO: 125 - protein, 339 aa, synthetic construct
MPENLKQPMPAAMQAYLTQLHQVIIPYLLAQGVKPNAINAREALANLTTANVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 126 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgaacgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 127 - protein, 339 aa, synthetic construct
MPENLKQPMPAAMQAYLTQLHQVIIPYLLAQGVKPNAINAREALANLTTAMVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 128 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgatggtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 129 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGCSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 130 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggctgttctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 131 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 132 - DNA, 1017 bp, synthetic construct
  source 1..1017: mol_type = other DNA
agcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggggattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcgctcgagtgagatccggctg

SEQ ID NO: 133 - protein, 339 aa, synthetic construct
MPENLKQPMPAAMQAYLTQLHQVIIPYLLAQGVKPNAINAREALANLTTAFVTDDPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 134 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgatccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 135 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMSGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 136 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatgagcggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 137 - protein, 339 aa, synthetic construct
MPENLKQPMPAAMQAYLTQLHQVIIPYLLAQGVKPNAINAREALANLTTAFVTDYPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 138 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgattatccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 139 - protein, 339 aa, synthetic construct
MPENLKQPMPAAMQAYLTQLHQVIIPYLLAQGVKPNAINAREALANLTTAFLTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 140 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttctgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 141 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSTIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 142 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgacgatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 143 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAEAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 144 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcagaggcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 145 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLETSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 146 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaacctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 147 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYYKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 148 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtactataaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 149 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 150 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtaccggaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 151 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLVKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 152 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttggtgaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 153 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGTITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 154 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtacgattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 155 - protein, 339 aa, synthetic construct
MPENLKQPMPAAMQAYLTQLHQVIIPYLLAQGVKPNAINAREALANLTTAFVTDSPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 156 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatagtccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 157 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDACAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 158 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcatgtgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 159 - protein, 339 aa, synthetic construct
MPENLKQPMPAAMQAYLTQLHQVIIPYLLAQGVKPNAINAREALANLTTAFVTPAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 160 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactccggccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 161 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYVKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 162 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacgtgaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 163 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIEASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 164 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatcgaggcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 165 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLEGSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 166 - DNA, 1017 bp, synthetic construct
  source 1..1017: mol_type = other DNA
agcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaggttcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcgctcgagtgagatccggctg

SEQ ID NO: 167 - protein, 339 aa, synthetic construct
MPENLKQPMPAAMQAYLTQLHQVIIPYLLAQGVKPNAINAREALANLTTAFVTDIPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYASLDYTMSQPSIEENGTGYLLESSKIRWYFDNYFSAYDDRELISPFWTDMATLLHHSPYQAPKTLHLTAGYCPLRDEDIGYHEQLQKAGAPSQLVHFADMPHAFLNMENLCPEQCKALYQKIGEFCQA

SEQ ID NO: 168 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatattccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 169 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGGMAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 170 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccgttttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 171 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 172 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 173 - protein, 339 aa, synthetic construct
MPENLKQPMPAAMQAYLTQLHQVIIPYLLAQGVKPNAINAREALANLTTAFVTDRPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIKWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 174 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatcgtccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatcaaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 175 - protein, 339 aa, synthetic construct
MPENLKQPMPAAMQAYLTQLHQVIIPYLLAQGVKPNAINAREAYANLTTAFVTDAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSTIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 176 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagcctatgcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatccgctggtacttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 177 - protein, 339 aa, synthetic construct
MPENLKQPMPAAMQAYLTQLHQVIIPYLLAQGVKPNAINAREAWANLTTAFVTFAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSTIEWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 178 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagcctgggcgaacttaaccacggcgtttgtgacttttgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 179 - protein, 339 aa, synthetic construct
MPENLKQPMPAAMQAYLTQLHQVIIPYLLAQGVKPNAINAREAYANLTTAFVTDAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSTIRWFFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 180 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagcctatgcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatccgctggttcttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 181 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHTAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIRWFFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 182 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcataccgctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatccgctggttcttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 183 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSKIRWFFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 184 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggttcttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 185 - protein, 339 aa, synthetic construct
MPENLKQPMPAAMQAYLTQLHQVIIPYLLAQGVKPNAINAREAYANLTTAFVTDAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIKWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 186 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagcctatgcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatcaaatggtatttcgacaattacttcagcgcgtacgatgaccgggaactgatcagcccattttggacagatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 187 - protein, 339 aa, synthetic construct
MPENLKQPMPAAMQAYLTQLHQVIIPYLLAQGVKPNAINAREAYANLTTAFVTDRPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 188 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagcctatgcgaacttaaccacggcgtttgtgactgatcgtccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatccgctggtacttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 189 - protein, 339 aa, synthetic construct
MPENLKQPMPAAMQAYLTQLHQVIIPYLLAQGVKPNAINAREAYANLTTAFVTDAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIEWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 190 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagcctatgcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 191 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIRWFFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 192 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggttcttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 193 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVFDKLYKKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIKWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 194 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtcttcgacaagttgtacaaaaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatcaaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 195 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVYDKLYQKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSKIRWFFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 196 - DNA, 1023 bp, synthetic construct
  source 1..1023: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccagaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggttcttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcgctcgag

SEQ ID NO: 197 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHVAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIEWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 198 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatgtggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 199 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHVAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 200 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatgtggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 201 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 202 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtacttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 203 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIEWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 204 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 205 - protein, 339 aa, synthetic construct
MPENLKQPMPAAMQAYLTQLHQVIIPYLLAQGVKPNAINAREALANLTTAFVTFAPPMAKIIDTVLVTPSNFRGYNVPLRLFVPSNCPLPNEQATADVPVMVYFHGGGHVAGSVTVYDKLYRKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 206 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgacttttgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatgtggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 207 - protein, 339 aa, synthetic construct
MPENLKQPMPAAMQAYLTQLHQVIIPYLLAQGVKPNAINAREAWANLTTAFVTDAPPMAKIIDTVLVTPSNFRGYNVPLRLFVPSNCPLPNEQATADVPVMVYFHGGGHTTGSVTVYDKLYQKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 208 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagcctgggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcataccactggcagtgtaaccgtctacgacaagttgtaccaaaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 209 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVYDKLYKKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 210 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctatgacaagttgtacaaaaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 211 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVYDKLYRKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSKIEWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 212 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 213 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVFDKLYQKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIEWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 214 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctttgacaagttgtaccaaaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 215 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHTAGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 216 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcataccgctggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 217 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVYDKLYKKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIEWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 218 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtacaaaaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 219 - protein, 339 aa, synthetic construct
MPENLKQPMPAAMQAYLTQLHQVIIPYLLAQGVKPNAINAREALANLTTAFVTFAPPMAKIIDTVLVTPSNFRGYNVPLRLFVPSNCPLPNEQATADVPVMVYFHGGGHMAGSVTVFDKLYKKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 220 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgacttttgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctttgacaagttgtacaagaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 221 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHTTGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 222 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatgtgactggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 223 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHVAGSVTVFDKLYKKLAEATGCVVVAPEYRLAPENRYPAAIDDAHAILTYLEATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 224 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatgtggctggcagtgtaaccgtcttcgacaagttgtacaaaaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 225 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHVTGSVTVFDKLYKKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 226 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatgtgactggcagtgtaaccgtcttcgacaagttgtacaaaaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 227 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHVTGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIKWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 228 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatgtgactggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatcaaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 229 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHVTGSVTVYDKLYRKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 230 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatgtgactggcagtgtaaccgtctacgacaagttgtaccgtaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatccgctggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 231 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMTGSVTVYDKLYQKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSKIEWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 232 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatgactggcagtgtaaccgtctacgacaagttgtaccaaaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgaaaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 233 - protein, 339 aa, synthetic construct
MPENLKQPMPAAMQAYLTQLHQVIIPYLLAQGVKPNAINAREALANLTTAFVTFAPPMAKIIDTVLVTPSNFRGYNVPLRLFVPSNCPLPNEQATADVPVMVYFHGGGHMAGSVTVYDKLYQKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIEWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 234 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgacttttgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctacgacaagttgtaccaaaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 235 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHVAGSVTVFDKLYKKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIEWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 236 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatgtggctggcagtgtaaccgtctttgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatcgaatggtatttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 237 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVYDKLYQKLAEATGCVVVAPEYRLAPENRYPAAIDDAHAILTYLEATLQQVGYACNGKLIIAGDSAGGAITATLVQDWLANRVSTDLAITHQILIYPSLDKTMSQPSIEENGTGYLLEKSKIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 238 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctatgacaagttgtaccaaaaattagcggaagcgactgggtgtgtggttgttgctccggaataccgcttagcaccggaaaaccgttatcctgcagccattgatgatgcacatgcgattctcacctatttggaagctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggacaaaacgatgagccaaccctccatcgaggagaatggcactgggtatctgctggaaaaatcgaaaatccgctggtacttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 239 - protein, 339 aa, synthetic construct
MPENLKQPMPAAMQAYLTQLHQVIIPYLLAQGVKPNAINAREALANLTTAFVTDAPPMAKIIDTVLVTPSNFRGYNVPLRLFVPSNCPLPNEQATADVPVMVYFHGGGHMAGSVTVFDKLYKKLAEATGCVVVAPEYRLAPENRYPAAIDDAHAILTYLTATLQQVGYACNGKLIIAGDSAGGAITATLVQDWLANRVSTDLAITHQILIYPSLDYTMSQPSIEENGTGYLLEKSTIRWYFDNYFSAYDDRELISPFWTDMATLLHHSPYQAPKTLHLTAGYCPLRDEGIGYHEQLQKAGAPSQLVHFADMPHAFLNMENLCPEQCKALYQKIGEFCQA

SEQ ID NO: 240 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgccgatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtcatatggctggcagtgtaaccgtctttgacaagttgtacaaaaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgccaccctggttcaggattggctggcgaatcgcgtttccacagatctggccattacgcaccagatcctgatctacccgtctctggactacacgatgagccaaccctccatcgaggagaatggcactgggtatctgctggaaaaatcgacaatccgctggtacttcgacaattacttcagcgcgtacgatgaccgggaactgatcagcccattttggaccgatatggctaccttactgcaccatagtccgtatcaagctccgaaaacgttacaccttaccgccggctattgccctcttcgtgatgagggcattgggtaccacgaacagctgcagaaagcgggtgcgccttcacagcttgtccatttcgccgatatgccgcatgcgttcctcaacatggagaacctttgtccggaacagtgcaaagcgctgtatcagaaaattggcgagttttgccaagcg

SEQ ID NO: 241 - protein, 339 aa, synthetic construct
MPENLKQPMPAAMQAYLTQLHQVIIPYLLAQGVKPNAINAREALANLTTAFVTDAPNMAKIIDTVLVTPSNFRGYNVPLRLFVPSNCPLPNEQATADVPVMVYFHGGGGMAGSVTVYDKLYKKLAEATGCVVVAPEYRLAPENRYPAAIDDAHAILTYLTATLQQVGYACNGKLIIAGDSAGGAITASLVQDWLANRVSTDLAITHQILIYASLDYTMSQPSIEENGTGYLLESSKIRWYFDNYFSAYDDRELSSPFWTDMATLLHHSPYQAPKTLHLTAGYCPLRDEDIGYHEQLQKAGALSELVHFADMPHAFLNMENLCPEQCKALYQKIGEFCQA

SEQ ID NO: 242 - DNA, 1017 bp, synthetic construct
  source 1..1017: mol_type = other DNA
atgccggaaaacctgaaacagcccatgccggccgctatgcaagcatatctgacgcagttgcatcaggtgatcattccatatctgctggcgcaaggtgtcaaaccaaatgcgattaacgcacgtgaagccctggcgaacttaaccacggcgtttgtgactgatgccccgaatatggcgaagatcattgatacggtcctcgtgactccgtcaaactttcgcggctataacgttccattgcgcctctttgtaccgtcgaactgtccactgcccaatgaacaggcaacagccgacgtacctgtgatggtgtactttcatggaggaggtggtatggctggcagtgtaaccgtctacgacaagttgtacaagaaattagcggaagcgactgggtgtgtggttgttgctccggaataccgcttagcaccggaaaaccgttatcctgcagccattgatgatgcacatgcgattctcacctatttgaccgctacactgcaacaggtgggctatgcctgcaatggcaaactgattatcgcaggcgattctgcaggtggtgccattaccgcctcgctggttcaggattggctggcgaatcgcgtttccacagatctggccattacgcaccagatcctgatctacgcatctctggactacacgatgagccaaccctccatcgaggagaatggcactgggtatctgctggaaagctcgaaaatccgctggtatttcgacaattacttcagcgcgtacgatgaccgggaactgagcagcccgttttggaccgatatggctaccttactgcaccatagtccgtatcaagctccgaaaacgttacaccttaccgccggctattgccctcttcgtgatgaggacattgggtaccacgaacagctgcagaaagcgggtgcgctttcagagcttgtccatttcgccgatatgccgcatgcgttcctcaacatggagaacctttgtccggaacagtgcaaagcgctgtatcagaaaattggcgagttttgccaagcg